(19) United States
(12) Patent Application Publication
(10) Pub. No.: US 2024/0047007 A1
(43) Pub. Date: Feb. 8, 2024

(54) METHODS, DEVICES, AND SYSTEMS FOR DETERMINING LOW GRADE GLIOMA (LGG) SUBTYPES IDENTIFIED THROUGH MACHINE LEARNING

(71) Applicant: THE REGENTS OF THE UNIVERSITY OF CALIFORNIA, Oakland, CA (US)

(72) Inventor: Hang CHANG, Moraga, CA (US)

(21) Appl. No.: 18/363,344

(22) Filed: Aug. 1, 2023

Related U.S. Application Data
(60) Provisional application No. 63/369,982, filed on Aug. 1, 2022.

(51) Int. Cl. G16B 20/00; C12Q 1/6886; G01N 33/574; G16B 40/30; G16H 50/20; G16H 20/40

(52) U.S. Cl. CPC G16B 20/00; C12Q 1/6886; G01N 33/57407; G16B 40/30; G16H 50/20; G16H 20/40

(57) ABSTRACT

A method for determining a Lower Grade Glioma (LGG) subtype for a subject. A device for determining an LGG subtype in a subject. A system using machine learning for determining a Lower Grade Glioma (LGG) subtype in a subject.

### CROSS-REFERENCE TO RELATED APPLICATIONS

This application claims priority to U.S. Provisional Patent Application Ser. No. 63/369,982, filed Aug. 1, 2022, which is hereby incorporated by reference.

### STATEMENT OF GOVERNMENTAL SUPPORT

The invention described and claimed herein was made utilizing funds supplied by the U.S. Department of Energy under Contract No. DE-AC02-05CH11231. The government has certain rights in this invention.

### FIELD OF THE INVENTION

The present invention is in the field of low grade glioma (LGG).

### BACKGROUND OF THE INVENTION

Gliomas is the most common primary central nervous system (CNS) malignant tumor, accounting for ˜80% of all CNS malignancies.¹ According to the 2007 WHO classification, gliomas were categorized into grades 1-4.² The 2021 WHO classification³ introduced a paradigm shift in the classification of CNS tumors combining histopathologic and genotypic features⁴ to reveal an “integrated” diagnosis. Factors affecting overall survival (OS) include age >40 years, astrocytic subtype, tumor maximum diameter >6 cm, tumors crossing the midline, and the patient's degree of neurological impairment, Karnofsky performance score, multiple lesions, IDH-mutant status, lpl9q status, TERT mutation status, and ATRX mutation status.⁵⁻⁸ Moreover, lower-grade gliomas (LGGs) are highly heterogeneous both at histopathological and molecular levels,^{4,9} resulting in significant variability in clinical outcomes.^{9,10} Therefore, to personalize care and treatment of LGG patients, accurate and robust patient stratification, which is significantly associated with clinical outcomes, is mandatory.

Cellular morphometric properties play key roles in cancer diagnosis and prognosis together with important molecular factors. Recently, deep neural networks (e.g., convolutional neural network [CNN]) have been successfully applied in several glioma-related studies.¹¹⁻¹³ However, the quantitative profiling and molecular association of the cellular morphometric landscape from whole-slide images (WSIs) remain inadequately investigated due to both technical and conceptual limitations.

### SUMMARY OF THE INVENTION

The present invention provides for a method for determining a Lower Grade Glioma (LGG) subtype for a subject. The present invention also provides for a device for determining an LGG subtype in a subject. The present invention also provides for a system using machine learning for determining a Lower Grade Glioma (LGG) subtype in a subject.

The present invention provides for a method for determining and treating a Lower Grade Glioma (LGG) subtype for a subject, the method comprising: (a) obtaining a tissue sample from a subject suffering from LGG, (b) determining a cellular morphometric subtype (CMS) and/or cellular morphometric biomarkers (CMBs) of the tissue sample, (c) identifying a LGG subtype of the subject as LGG subtype 1 or LGG subtype 2, and (d) treating the subject wherein (i) when the subject is LGG subtype 1, the subject is not treated with immunotherapy, and (ii) when the subject is LGG subtype 2, the subject is treated with immunotherapy.

In some embodiments, the sample comprises a tumor cell, T cell, B cell, and macrophages, and the like.

Cellular morphometric biomarkers (CMBs) are identified with artificial intelligence technique. Consensus clustering is used to define CMS. Survival analysis is performed to assess the clinical impact of CMBs and CMS. A nomogram is constructed to predict 3- and 5-year overall survival (OS) of LGG patients. Tumor mutational burden (TMB) and immune cell infiltration between subtypes are analyzed using the Mann-Whitney U test. In some embodiments, the CMB is extracted from a machine learning (ML) pipeline from whole-slide images of tissue histology, wherein the ML pipeline identifies and externally validates robust CMS of LGGs. In some embodiments, the method uses a framework (CMS-ML) for CMS discovery in LGG associated with specific molecular alterations, immune microenvironment, prognosis, and treatment response. In some embodiments, the cellular morphometric descriptors used are described in Table 1. The differentially expressed genes between Subtype 2 and Subtype 1 patients is described in Table 2.

Further detailed is described in Liu et al. “Clinical significance and molecular annotation of cellular morphometric subtypes in lower-grade gliomas discovered by machine learning,” Neuro. Oncol. 25(5):68-81, 2023, including the corresponding Supplementary Material; all of which is hereby incorporated by reference).

In some embodiments, the method for determining a Lower Grade Glioma (LGG) subtype for a subject comprises: (a) segment nuclear regions of a sample obtained from a subject; (b) measure the corresponding morphometric properties of the sample, such as the corresponding morphometric properties from hematoxylin and/or eosin (H&E) stained whole slide images of tissue histology of the sample; (c) apply pre-identified cellular morphometric biomarkers with prebuilt stacked predictive sparse decomposition (SPSD) model to construct cellular-level and patient-level morphometric representation; and (d) apply pre-built subtype model to stratify LGG subjects into subclasses.

In some embodiments, the LGG subjects are identified as LGG subtype 1 is treated with radiation therapy and/or chemotherapy. In some embodiments, the LGG subjects are identified as LGG subtype 2 is treated with radiation therapy and/or chemotherapy, and immunotherapy (anti-PD-1, anti-PD-L1, and/or anti-CTLA-4 immunotherapy).

Methods or means of administering anti-PD-1, anti-PD-L1, and/or anti-CTLA-4 immunotherapy is described by Wojtukiewicz et al., “Inhibitors of immune checkpoints—PD-1, PD-L1, CTLA-4—new opportunities for cancer patients and a new challenge for internists and general practitioners,”Cancer Metastasis Rev. 40(3):949-982 (2021); Ghouzlani et al., “Immune Checkpoint Inhibitors in Human Glioma Microenvironment,”Front. Immunol. 12: Article 679425, 2021; and, Deshmukh, “CTLA-4 and PD-L₁ or PD-₁ Pathways: Immune Checkpoint Inhibitors and Cancer Immunotherapy,”J. Cancer Immunol. 2(1):10-12, 2020; which are all herein incorporated by reference.

In some embodiments, the device for determining a Lower Grade Glioma (LGG) subtype in a subject comprises: (a) a means to obtain nuclear segmentation from whole slide image of tissue histology; (b) representation learning from cellular morphometric properties derived from segmented nuclei; and (c) subtype identification based on patient-level morphometric context representation.

Other objects, features, and advantages of the present invention will be apparent to one of skill in the art from the following detailed description and figures.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Graphical illustration of the study.

FIG. 2. Unsupervised feature learning discovers CMBs with clinical significance and molecular relevance. (A) Prognostic significant CMBs with favorable and unfavorable examples; (B-D) LGG patients within CMB-low and CMB-high groups show significant differences in various tumor microenvironmental factors (B), overall survival (C), and genetic instability (D).

FIG. 3. Lower-grade glioma (LGG) patient subtype provides significant and independent prognostic impact. (A) Consensus clustering model for LGG patient subtypes discovery and inference; (B-D) subtype-specific patients in TCGA-LGG, ZN-LGG, and SU-LGG cohorts form distinct clusters in patient-level cellular morphometric context space; (E-G) subtype-specific patients in TCGA-LGG, ZN-LGG, and SU-LGG cohort show significant difference in survival; (H-J) patient subtype in TCGA-LGG, ZN-LGG, and SU-LGG cohort is a significant and independent prognostic factor.

FIG. 4. Development and validation of nomogram predicting the 3- and 5-year survival of lower-grade glioma (LGG) patients. (A) Nomogram predicting the 3- and 5-year survival of LGG patients. (B) Calibration analysis at 3 years in the training set of TCGA-LGG cohort. (C) Calibration analysis at 5 years in the training set of TCGA-LGG cohort. (D) Calibration analysis at 3 years in the test set of TCGA-LGG cohort. (E) Calibration analysis at 5 years in the test set of TCGA-LGG cohort.

FIG. 5. (A) Patient subtypes in TCGA-LGG cohort show significant difference in various tumor microenvironmental factors. (B) Immunohistochemistry (IHC) staining confirms the significantly more infiltrating T cells (CD3+), B cells (CD20+), and macrophages M1 (CD80+) immune cells in subtype 2 LGG patients (scale bar=100 μm).

FIG. 6 Immunohistochemistry (IHC) staining confirms the upregulation of PD-1, PD-L1, and CTLA-4 in subtype 2 LGG patients. (A) Subtype-specific expression of PD-1 (first row), PD-L1 (second row), and CTLA-4 (third row) in TCGA-LGG cohort. (B) Representative examples of PD-1 staining (first row), PD-L1 staining (second row), and CTLA-4 staining (third row) in subtype 1 and 2 LGG patients (scale bar=100 μm), respectively, where PD-1 expression was frequently observed in the plasma of lymphocytes around blood vessels; PD-L1 was widely expressed in the membrane of tumor cells, while slightly in the cytoplasm; and CTLA-4 positive expression was majorly observed in the cytoplasm of lymphocytes around blood vessels. (C) Subtype-specific expression of PD-1 (first row), PD-L1 (second row), and CTLA-4 (third row) was quantified via IHC staining in ZN-LGG cohort.

FIG. 7. Patient subtypes in TCGA-LGG cohort show significant difference in tumor mutation burden and somatic copy number alteration (SCNA).

FIG. 8. The expression levels of other immune suppression genes between subtype 1 and subtype 2 patients.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to an “expression vector” includes a single expression vector as well as a plurality of expression vectors, either the same (e.g., the same operon) or different; reference to “cell” includes a single cell as well as a plurality of cells; and the like.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” as used herein means a value that includes 10% less and 10% more than the value referred to.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

The invention having been described, the following examples are offered to illustrate the subject invention by way of illustration, not by way of limitation.

### Example 1

### Clinical Significance and Molecular Annotation of Cellular Morphometric Subtypes in Lower-Grade Gliomas Discovered by Machine Learning

### Background

Lower-grade gliomas (LGG) are heterogeneous diseases by clinical, histological, and molecular criteria. We aimed to personalize the diagnosis and therapy of LGG patients by developing and validating robust cellular morphometric subtypes (CMS) and to uncover the molecular signatures underlying these subtypes.

### Methods

Cellular morphometric biomarkers (CMBs) were identified with artificial intelligence technique from TCGA-LGG cohort. Consensus clustering was used to define CMS. Survival analysis was performed to assess the clinical impact of CMBs and CMS. A nomogram was constructed to predict 3- and 5-year overall survival (OS) of LGG patients. Tumor mutational burden (TMB) and immune cell infiltration between subtypes were analyzed using the Mann-Whitney U test. The double-blinded validation for important immunotherapy-related biomarkers was executed using immunohistochemistry (IHC).

### Results

We developed a machine learning (ML) pipeline to extract CMBs from whole-slide images of tissue histology; identifying and externally validating robust CMS of LGGs in multicenter cohorts. The subtypes had independent predicted OS across all three independent cohorts. In the TCGA-LGG cohort, patients within the poor-prognosis subtype responded poorly to primary and follow-up therapies. LGGs within the poor-prognosis subtype were characterized by high mutational burden, high frequencies of copy number alterations, and high levels of tumor-infiltrating lymphocytes and immune checkpoint genes. Higher levels of PD-1/PD-L1/CTLA-4 were confirmed by IHC staining. In addition, the subtypes learned from LGG demonstrate translational impact on glioblastoma (GBM).

### Conclusions

We developed and validated a framework (CMS-ML) for CMS discovery in LGG associated with specific molecular alterations, immune microenvironment, prognosis, and treatment response.

### Importance of the Study

LGGs are highly heterogeneous both at the histopathological and molecular level reflected in significant variability in clinical outcomes. Therefore, to personalize care and treatment of LGG patients, accurate and robust patient stratification, which is significantly associated with clinical outcomes, is mandatory. In this study, we developed and multicentrically validated a framework (CMS-ML) for CMS discovery in LGG associated with specific molecular alterations, immune microenvironment, prognosis, and treatment response. And the subtypes learned from LGG demonstrate translational impact on glioblastoma. Our findings have potential clinical implications to facilitate precision diagnosis and personalized treatment of LGG patients. In addition, CMS-ML may provide potential clinical value across tumor types.

To capture the heterogeneous cytoarchitecture of gliomas, we developed a high-throughput and robust computational pipeline that quantifies tissue histology at the cellular level¹⁴ with applications to tumor classificationL and molecular association.¹⁶ In addition, we introduced stacked predictive sparse decomposition (SPSD)¹⁷ for mining underlying cellular morphometric properties within WSI. Here, we applied SPSD to LGG cohorts to discover clinically relevant cellular morphometric subtypes (CMSs) and evaluate the clinical impacts and molecular correlation of CMSs

### Method

Data Collection

The patient data in this retrospective study, including tissue histology diagnostic slides and the clinical information, were collected from TCGA-LGG cohort (Supplementary Table 1; Supplementary Tables and Figures can be found in U.S. Provisional Patent Application Ser. No. 63/369,982, filed Aug. 1, 2022, and Liu et al. “Clinical significance and molecular annotation of cellular morphometric subtypes in lower-grade gliomas discovered by machine learning,” Neuro. Oncol. 25(5):68-81, 2023; both of which are hereby incorporated by reference), Zhongnan Hospital of Wuhan University (ZN-LGG cohort, between January 2016 and May 2019, Supplementary Table 2), the Medical Center of Stanford University (SU-LGG cohort, between January 2013 and December 2014, Supplementary Table 3), TCGA-GBM cohort (Supplementary Table 4), and Zhongnan Hospital of Wuhan University (ZN-GBM cohort, between January 2016 and May 2019, Supplementary Table 5) to form the discovery cohort and multicenter validation cohorts. The inclusion criteria were primary LGG and GBM with diagnostic slides and OS information available. This study was approved by the institutional review board (IRB) of Zhongnan Hospital of Wuhan University, Stanford University, and Lawrence Berkeley National Laboratory, with a waiver of informed consent.

Treatment Response in TCGA-LGG Cohort

The treatment response in TCGA-LGG cohort was assessed using Response Evaluation Criteria in Solid Tumors (RECIST)¹⁸ as complete remission, partial remission, progressive disease, and stable disease. Here, we categorized patient response into Response (including complete/partial remission), and non-Response (including progressive/stable disease).

Identification of Cellular Morphometric Biomarkers

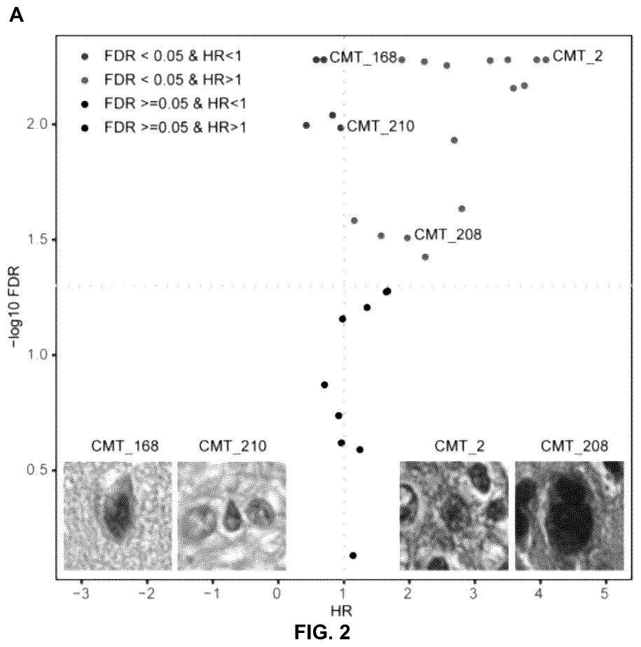

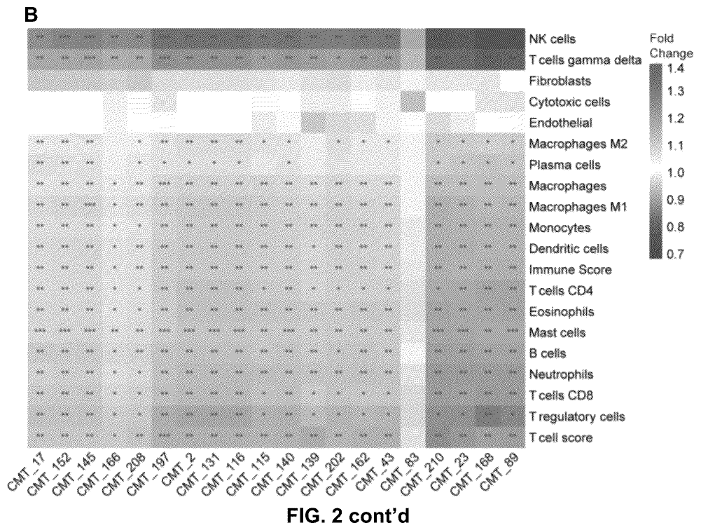

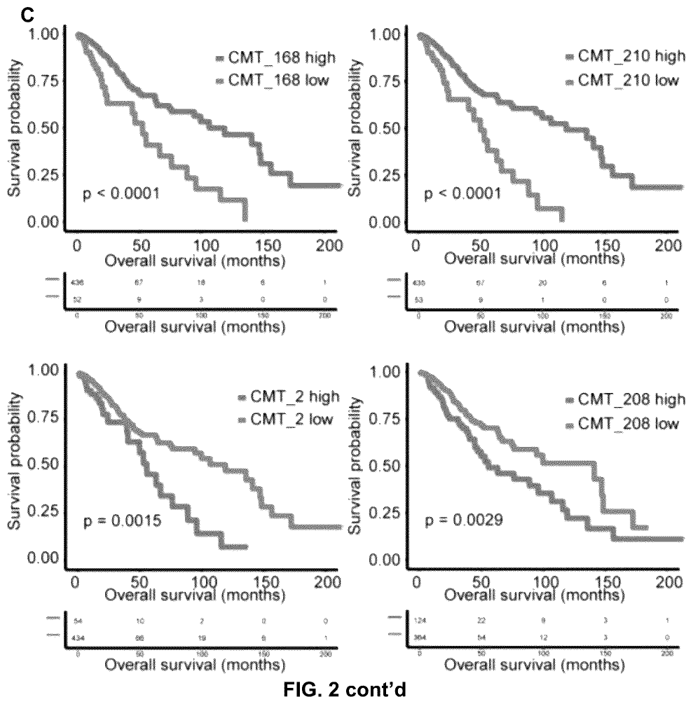

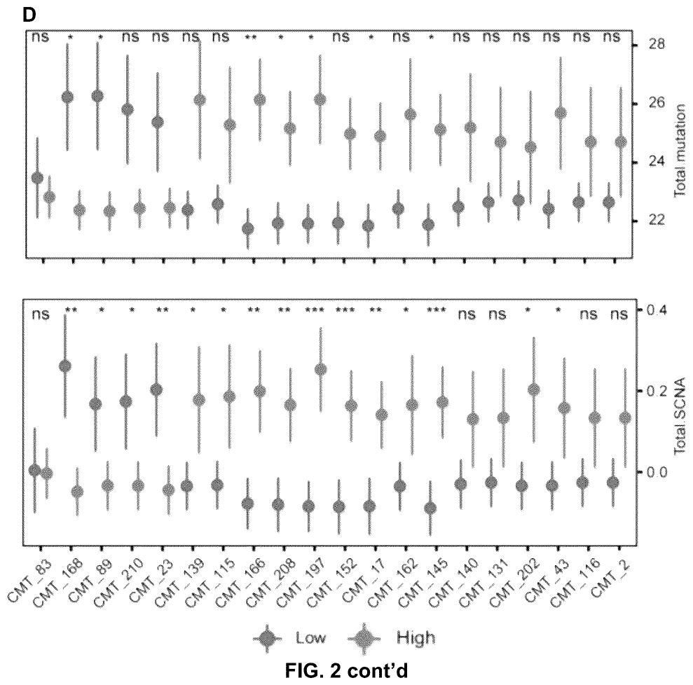

We developed an unsupervised machine learning pipeline based on SPSD¹⁷ for the discovery of underlying cellular morphometric characteristics from the 15 cellular morphometric features extracted from the WSIs of TCGA-LGG cohort (Supplementary Method 1). We then identified 256 cellular morphometric biomarkers (CMBs) for cellular object representation. Specifically, we used a single network layer with 256 dictionary elements (i.e., CMBs) and sparsity constraint 30 at a fixed random sampling rate of 1000 cellular objects per WSI from TCGA-LGG cohort (Supplementary FIG. 2A), where the network parameters (i.e., dictionary size and sparsity) were experimentally optimized to maintain the data reconstruction error ratio under certain threshold (i.e., 10% in this study, Supplementary FIGS. 2B and C). The pre-trained SPSD model reconstructed each cellular object as a sparse combination of pre-identified 256 CMBs, and thereafter represented it as the sparse code (i.e., reconstruction sparse coefficients), where the sparsity constraint enforced the reconstruction contribution mainly from the top 30 CMBs.

Clinical and Biological Evaluation of CMBs

We evaluated the prognostic impact of the top 30 CMBs with largest variations mined from TCGA-LGG cohort with Cox proportional hazards regression (CoxPH) model (survival package in R, Version 3.2-3), and examined the effects of high or low levels of each prognostic significant CMB on OS using Kaplan-Meier analysis (survminer package in R, Version 0.4.8) and log-rank test (survival package in R, Version 3.2-3), where TCGA-LGG cohort was divided into CMB-high and CMB-low groups per CMB (survminer package in R, Version 0.4.8). Meanwhile, we evaluated biological significance between these groups by assessing their relationship with factors available in TCGA-LGG cohort using the Mann-Whitney U test.

Construction of Patient-Level Cellular Morphometric Context Representation

The patient-level representation was constructed based on pre-identified 256 CMBs as an aggregation (i.e., max-pooling) of all the cellular sparse codes extracted via pre-built SPSD model from the cellular objects belonging to the same patients following these steps consecutively: (1) delineation of cellular architecture and extraction of cellular morphometric properties from WSIs of each patient; (2) construction of cellular sparse codes for the cellular objects belonging to each patient based on pre-identified 256 CMBs and pre-built SPSD model; (3) aggregation (i.e., max-pooling) of all cellular sparse codes belonging to the same patient to form the patient-level cellular morphometric representation; and (4) selection of the top 30 CMBs with the largest variations identified in TCGA-LGG cohort as the final patient-level cellular morphometric representation.

Identification and Application of CMS

The CMS was identified based on patient-level cellular morphometric context representation through consensus clustering¹⁹ (ConsensusClusterPlus R package, Version 1.50.0) with hierarchical clustering, Pearson's correlation, and 500 bootstrapping iterations; and the optimal number of subtypes was determined by the consistency of cluster assignment (consensus matrix) and the prognostic impact of subtypes. For a new patient, the subtype was assigned as follows: (1) construct patient-level cellular morphometric context representation with pre-built CMBs and SPSD model; (2) calculate the Pearson's distances between the new patient's representation and the mean representation of each pre-identified patient subtype; and (3) assign the new patient to its closest subtype yielding smallest Pearson's distance.

Clinical Evaluation and Validation of CMS

We evaluated and independently validated the clinical impact of pre-identified CMSs from TCGA-LGG cohort, ZN-LGG cohort, SU-LGG cohort, TCGA-GBM cohort, and ZN-GBM cohort, respectively. Refer to Supplementary Method 2 for details.

Differences in Gene Expression, Mutation Load, and Immune Microenvironment Between CMSs

We evaluated the differences in gene expression, mutation load, and immune microenvironment between CMSs. Refer to Supplementary Methods 3 for details.

Immunohistochemistry Staining

Immunohistochemistry (IHC) staining was carried out on 4-μm sections of formalin-fixed and paraffin-embedded tissues according to standard protocols (see Supplementary Method 4 for details).

Statistical Analysis

Refer to Supplementary Method 5 for details.

### Results

Study Design and Characteristics of Patient Cohorts

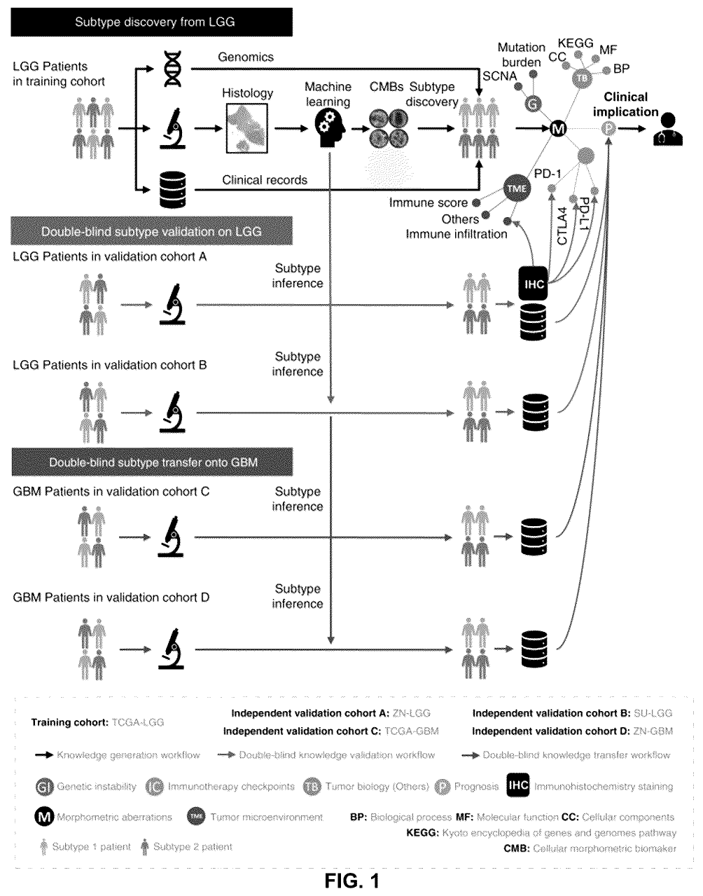

We used three retrospective LGG cohorts to evaluate and independently validate the prognostic impact of CMSs; and used two retrospective GBM cohorts to evaluate the generalizability and translational impact of LGG-driven CMSs in GBM (FIG. 1). The TCGA-LGG cohort served as discovery set including 488 LGG patients. There were 271 (55.5%) male and 217 (44.5%) female patients, with a median age of 41 years (range: 14-87 years). The ZN-LGG cohort included 70 LGG patients, where 36 patients (51.4%) were male and 34 (48.6%) were female. Median age was 47.0 years (range: 6-72 years). The SU-LGG cohort included 37 LGG patients, where 22 patients (59.5%) were male and 15 (40.5%) were female, and the median age was 41.0 years (range: 1-83 years). The TCGA-GBM cohort included 380 GBM patients, where 145 patients (38.2%) were male and 234 (61.6%) were female and the median age was 59.0 years (range: 10-89 years). The ZN-GBM cohort included 77 GBM patients, where 23 patients (29.9%) were male and 53 (68.8%) were female and the median age was 56.0 years (range: 5-81 years).

Identification of CMBs Using Unsupervised Representation Learning

Our pipeline¹⁴ recognized and delineated over 400 million cellular objects from TCGA-LGG chort; over 25 million cellular objects from ZN-LGG cohort; over 10 million cellular objects from SU-LGG cohort; over 400 million cellular objects from TCGA-GBM cohort; and over 25 million cellular objects from ZN-GBM cohort, where each cellular object was represented with 15 morphometric properties (Supplementary FIG. 1A, Supplementary Table 6, Supplementary Method 1).

Next, we trained SPSD¹⁷ model based on pre-quantified cellular objects randomly selected from TCGA-LGG cohort to discover the CMBs (Supplementary FIG. 2). After training, the pre-built SPSD model reconstructed each cellular object as a sparse combination of the pre-identified 256 CMBs, which led to the novel representation of each single cellular object as the 256 sparse codes. Thereafter, the corresponding 256-dimensional cellular morphometric context representation of each patient was an aggregation (Supplementary FIG. 1B) of all delineated cellular objects belonging to that patient (Supplementary Tables 7-11). The final patient-level cellular morphometric context representation was optimized by using the top 30 CMBs with the largest variations (sparsity constraint of SPSD model), which contributed to 98.84% of the total data variations.

Clinical and Biological Evaluation of CMBs

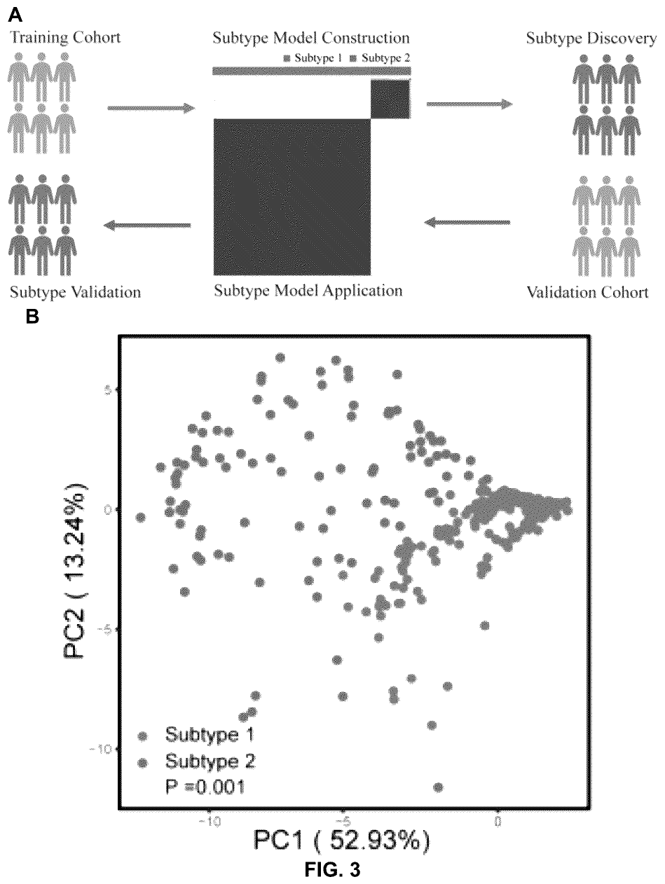

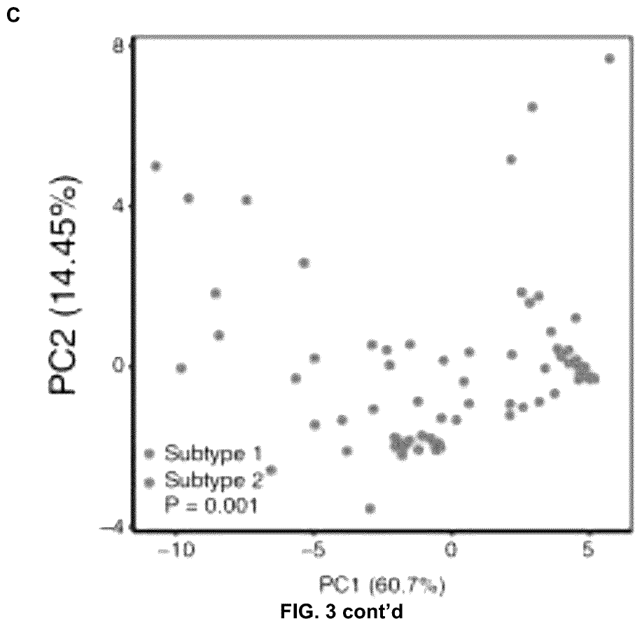

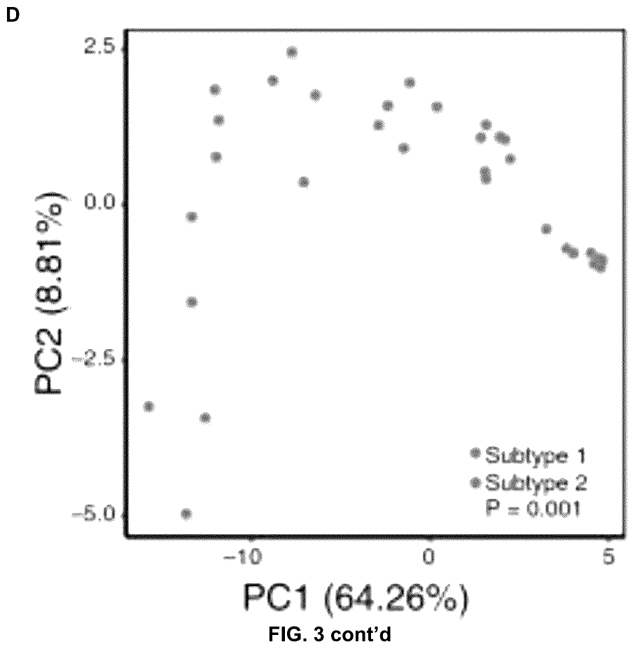

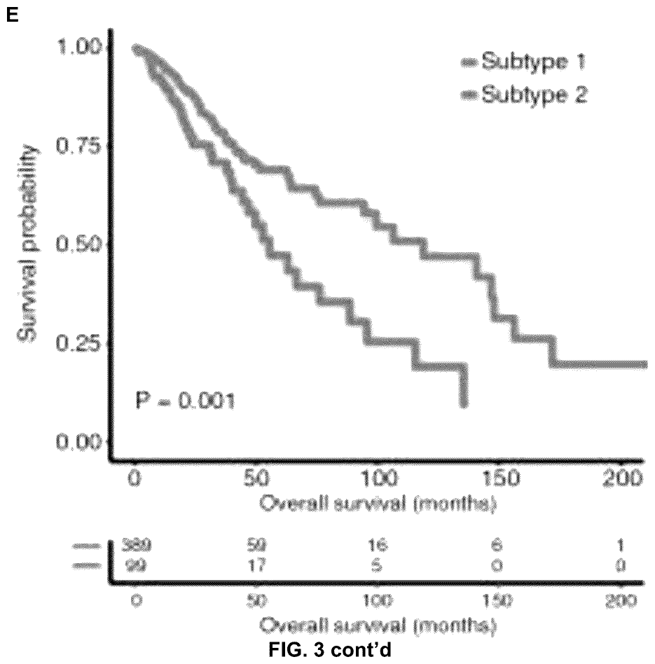

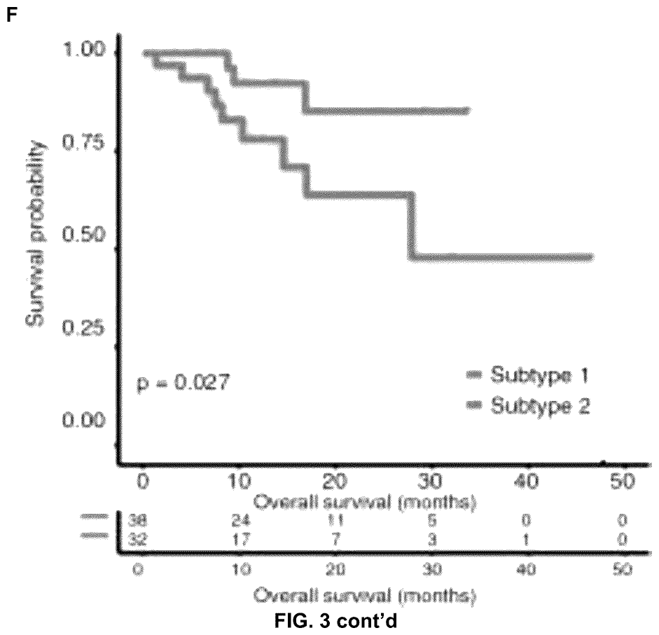

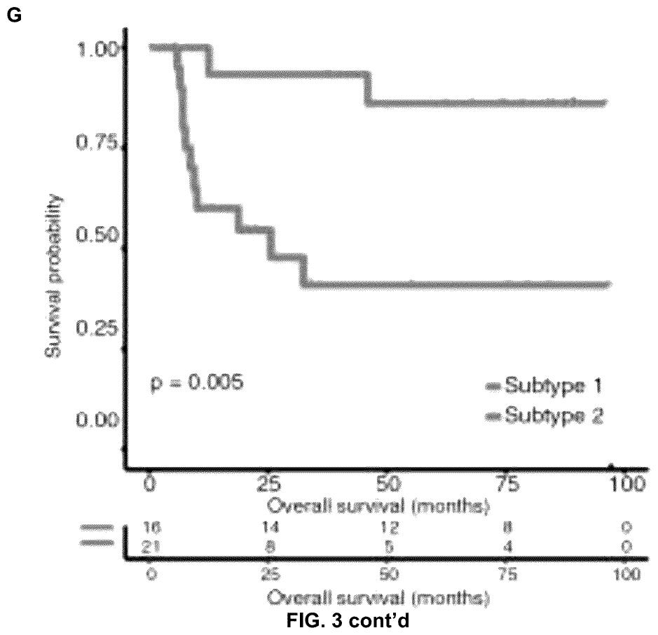

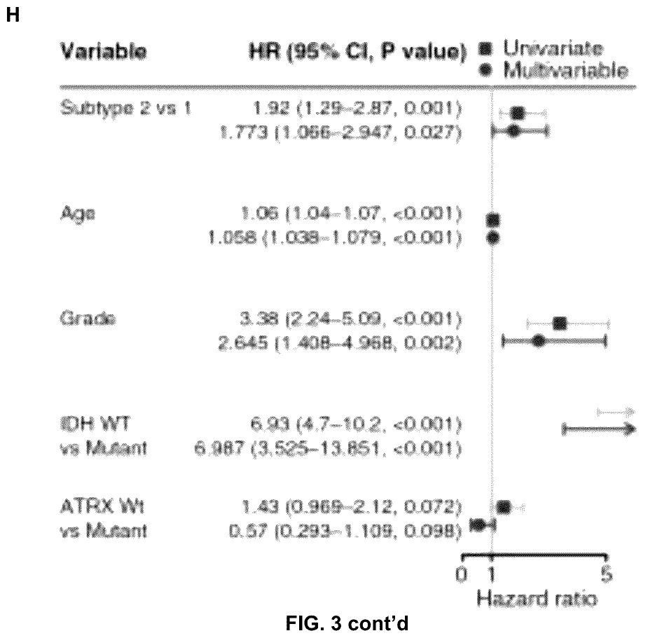

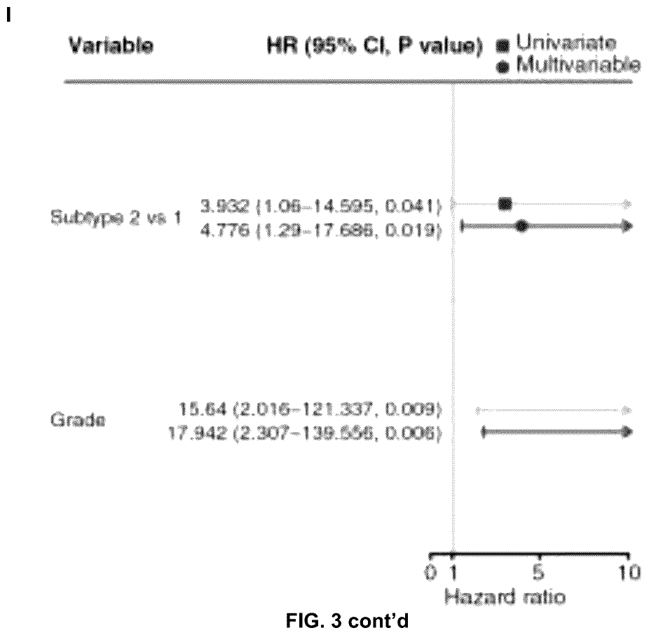

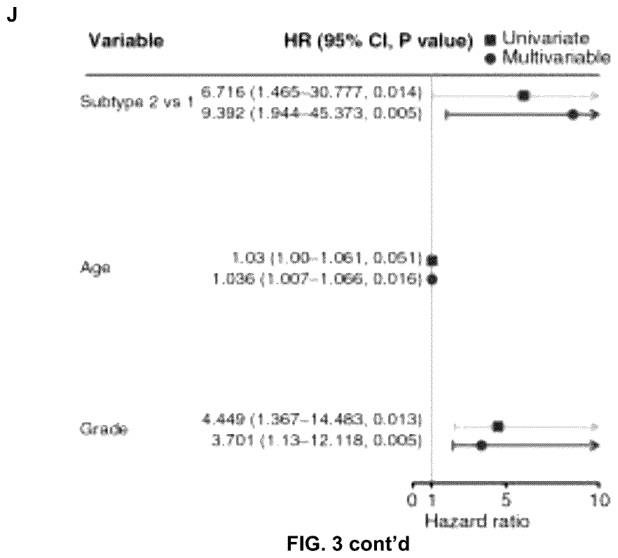

We next evaluated the association of the 30 CMBs with respect to histological meanings, prognosis, and cancer biology. Our survival analysis revealed that 20 CMBs had significant prognostic impact (false discovery rate [FDR]<0.05), where 5 of them were prognostically favorable (hazard ratio [HR]<1) and 15 prognostically unfavorable (HR>1) (FIG. 2A, Supplementary FIG. 3, Supplementary Table 12). Examples of prognostically significant CMBs (FIG. 2, Panel A, Supplementary FIG. 3) demonstrated the capability of our pipeline in acquiring biomedically meaningful and interpretable histopathological cellular concepts (Supplementary Table 13). For example, these CMBs captured atypical nuclear contour (e.g., CMB_139, CMB_115, CMB_152, CMB_131), nuclear pleomorphism with increasing variation in nuclear size, shape (e.g., CMB_208) or multinucleated tumor cells (e.g., CMB_145), etc.

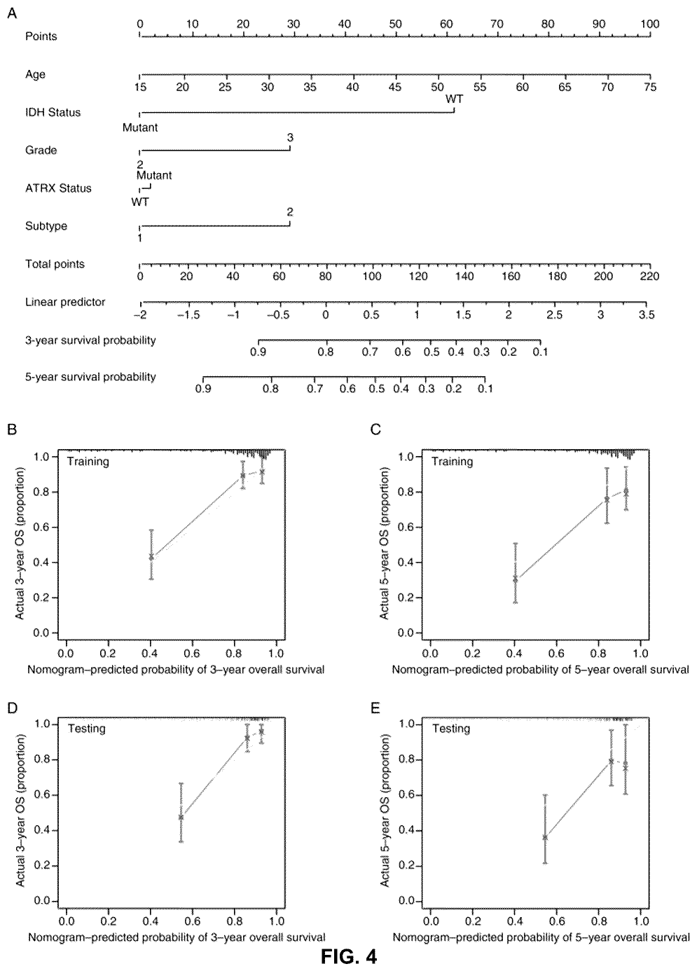

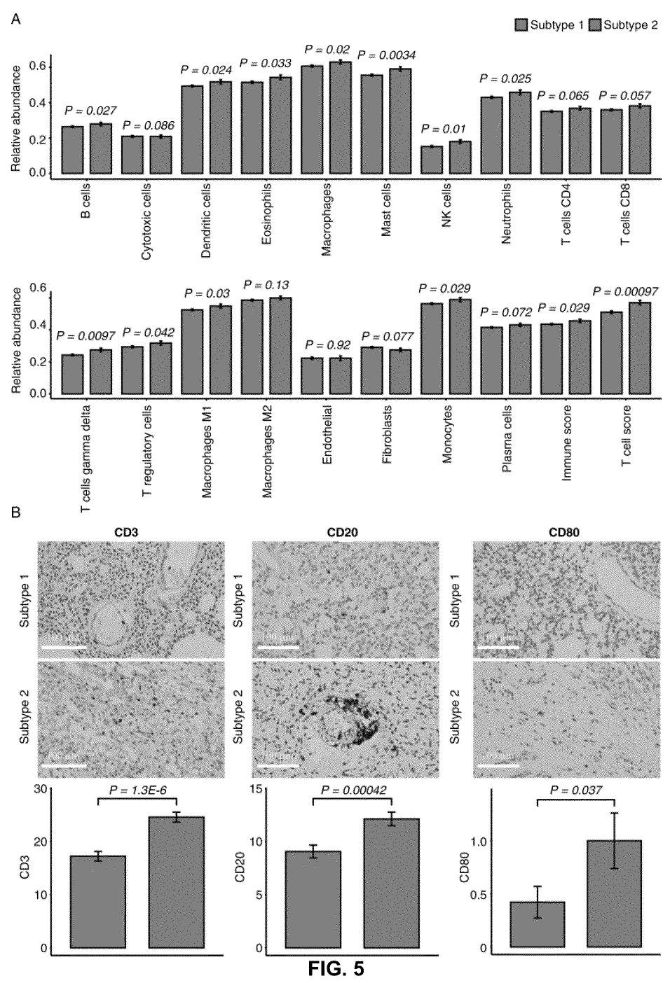

Additionally, the TCGA-LGG patient cohort was divided into two groups based on each CMB. The Kaplan-Meier curves showed significant impact (P<0.01, FIG. 2, Panel B, Supplementary FIG. 4) of the levels of each CMB on OS. Thereafter, we evaluated biological significance between patient groups with high and low CMB levels in the TCGA-LGG cohort and discovered significant correlations (P<0.05) with tumor microenvironment factors, including the relative abundance of tumor immune cells and fibroblast,²⁰ and predictors of immunotherapy response (FIG. 2, Panel C, Supplementary FIGS. 5 and 6). Levels of prognostically favorable CMBs correlated negatively, whereas levels of prognostic unfavorable CMBs correlated positively with tumor-infiltrating immune cells and the expression levels of PD-1 and PD-L1, but not to fibroblasts (P>0.05; FIG. 2, Panel C, Supplementary FIGS. 5 and 6). Finally, we detected a significant correlation between focal somatic copy number alteration (SCNA) and tumor mutational burden (TMB) (P<0.05; FIG. 2, Panel D).

Identification and Validation of CMS

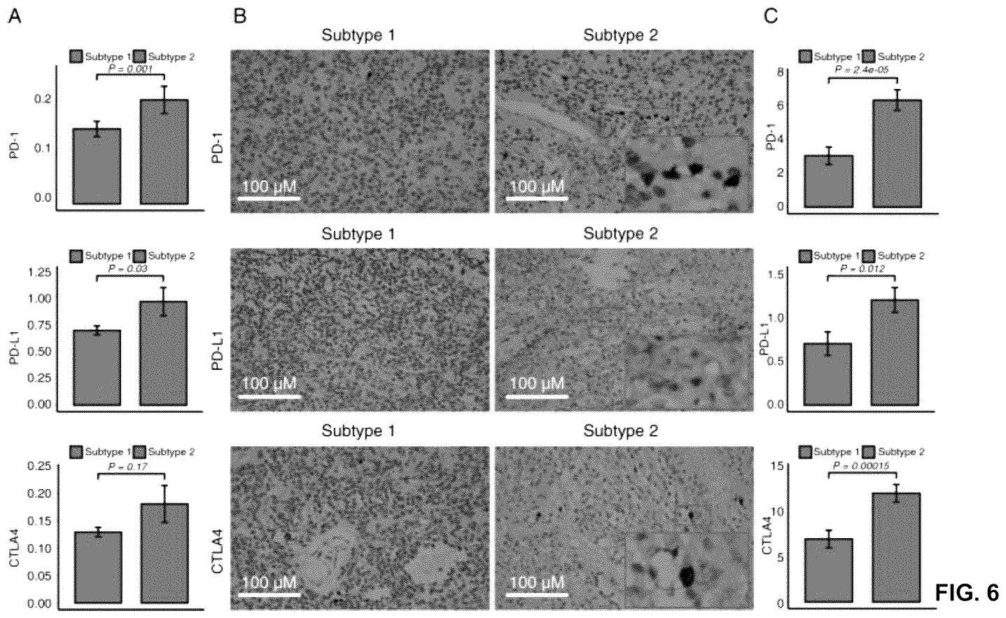

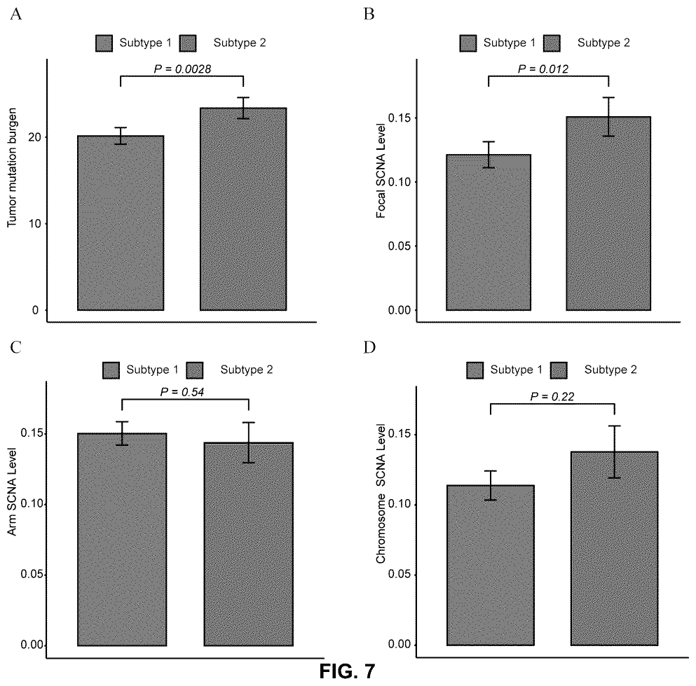

Consensus cluster analysis using 30 CMBs identified three CMSs from TCGA-LGG cohort with significantly differing prognosis (log-rank P<0.0001; Supplementary FIG. 7). Given the small number of patients (n=4) in subtype 3, as well as its prognostic similarity to subtype 2 patients, we merged subtypes 3 and 2, and referred this combination as subtype 2 in the rest of this study (FIG. 3, Panel A). Accordingly, the TCGA-LGG cohort contained 389 subtype 1 and 99 subtype 2 patients. The patient-level cellular morphometric context representation in TCGA-LGG cohort formed significantly distinct clusters (P=0.001, FIG. 3, Panel B). Importantly, two CMSs, predicted with pre-built subtype model, were portioned in two validation sets. Specifically, ZN-LGG cohort was stratified into subtype 1 (38 patients) and subtype 2 (32 patients), whereas SU-LGG cohort was stratified into subtype 1 (16 patients) and subtype 2 (21 patients). Moreover, the patient-level representation in both validation cohorts also formed significantly distinct clusters (P=0.001, FIG. 3, Panels C and D).

Clinical Significance of CMSs

We examined the association between CMSs and clinical and tumor characteristics in TCGA-LGG cohort. Surprisingly, there was no significant association between CMSs and any clinical/molecular prognostic factors (including age, grade, histological type, IDH mutation status, 1p/19q codeletion, MGMT promoter status, TERT promoter status, and ATRX status) (Supplementary Table 1). This finding was confirmed in both validation cohorts (Supplementary Tables 2 and 3).

In the TCGA-LGG cohort where genetic alteration burden information was available, Maftool analysis showed significantly higher TMB (P=0.003) and focal SCNA score (P=0.012) in subtype 2 patients (FIG. 7), indicating a higher level of genomic instability of tumors from subtype 2.

Kaplan-Meier analysis showed significantly shorter OS of subtype 2 than subtype 1 patients (P=0.001, FIG. 3, Panel E). Furthermore, univariate and multivariate CoxPH models indicated the independent prognostic impact of CMSs in TCGA-LGG cohort after adjusting for other significant clinical and molecular factors, including age, histological type, grade, IDH mutation status, and ATRX mutation status (HR: 1.773, 95% CI: 1.066-2.947, P=0.027; FIG. 3, Panel H, Supplementary Table 14). The combination of CMSs and clinical and molecular factors provided significantly improved (P<0.001, Supplementary FIG. 9) prediction of OS (median C-index: 0.860, 95% CI: 0.859-0.861) compared to classical models with only clinical and molecular factors (median C-index: 0.857, 95% CI: 0.856-0.858). Moreover, the nomogram (FIG. 4, Panel A), built upon patient subtype and clinical and molecular factors, significantly correlated with OS of TCGA-LGG patients, and provided excellent prediction [C-indexes for validation on the training set and testing set with 1000 bootstraps were 0.8334 (95% CI: 0.8322-0.8345) and 0.8014 (95% CI: 0.8001-0.8026), respectively] of the 3- and 5-year OS of TCGA-LGG patients, which was further confirmed by calibration analysis on the training (FIG. 4, Panels B and C) and testing set (FIG. 4, Panels D and E), respectively. Meanwhile, a dynamic nomogram further facilitated its potential clinical implications at: https://liuxiaoping.shinyapps.io/LGG nomogram. Additionally, the chi-square test showed significantly poor response of subtype 2 patients with respect to primary therapy (P<0.001) and follow-up treatment (P=0.002) (Supplementary Table 1).

Importantly, the double-blind deployment of the pre-built CMS model on both validation cohorts with independent survival analysis confirmed the significantly worse OS of subtype 2 patients (P=0.027 in ZN-LGG, P=0.005 in SU-LGG, FIG. 3, Panels F and G). Furthermore, univariate and multivariate CoxPH models confirmed the independent prognostic impact of CMSs after adjustment for other significant clinical factors in both validation cohorts (ZN-LGG:HR: 4.776, 95% CI: 1.29-17.686, P=0.019; SU-LGG:HR: 9.392, 95% CI: 1.944-45.373, P=0.005; FIG. 3, Panels I and J, Supplementary Tables 15 and 16).

Interestingly, the direct translation of the pre-built CMS model on TCGA-GBM and ZN-GBM cohorts confirmed the clinical impact of CMS learned from LGG on GBM patients (Supplementary FIG. 10). Consistent with our observations on LGG cohorts, GBM patients in both cohorts were stratified into distinct clusters (P=0.001 in TCGA-GBM; P=0.001 in ZN-GBM; Supplementary FIGS. 10A and B), and the subtype 2 GBM patients demonstrated significantly worse OS compared with subtype 1 GBM patients (P=0.00051 in TCGA-GBM; P<0.001 in ZN-GBM; Supplementary FIGS. 10C and D). Furthermore, univariate and multivariate CoxPH models confirmed the independent prognostic impact of CMSs in GBM patients after adjusting for significant clinical/molecular factors in both GBM cohorts (TCGA-GBM-HR: 1.457, 95% CI: 1.002-2.117, P=0.049; ZN-GBM-HR: 3.101, 95% CI: 2.006-7.491, P<0.001; Supplementary FIGS. 10E and F, Supplementary Tables 17 and 18). Furthermore, restricted mean survival time (RMST)²¹ analysis on both LGG and GBM patients (Supplementary Table 19) suggested the difference in follow-up times across cohorts had no significant influence on the prognostic value of CMS.

Lastly, we performed pooled analysis combing all LGG and GBM patients into Pooled-LGG (595 patients) and Pooled-GBM (457 patients) cohorts, respectively. The pooled analysis confirmed (1) the significantly distinct stratification of patients (Pooled-LGG: P=0.001, Supplementary FIG. 11A; Pooled-GBM: P=0.001, Supplementary FIG. 12A); (2) the significantly worse OS of subtype 2 patients (Pooled-LGG: P<0.001, Supplementary FIG. 11B; Pooled-GBM: P<0.001, Supplementary FIG. 12B); and (3) the independent prognostic impact of CMSs in both pooled cohorts (Pooled-LGG-HR: 2.315, 95% CI: 1.617-3.315, P<0.001, Supplementary FIG. 11C, Supplementary Table 20; Pooled-GBM HR: 1.57, 95% CI: 1.206-2.044, P=0.001, Supplementary FIG. 12C, Supplementary Table 21). Interestingly, OS difference between LGG subtypes was independent of tumor grade (Grade2: P=0.037; Grade3: P<0.0001; Supplementary FIG. 11D) and histology types (Astrocytoma: P=0.0046, Oligodendroglioma: P=0.012, Oligoastrocytoma: P=0.0013; Supplementary FIG. 11E), further demonstrating the independent clinical value of CMSs.

Molecular Annotation Underlying CMSs

To gain insight into molecular differences underlying CMSs, we used available transcriptome data from TCGA-LGG and identified 316 differentially expressed genes (DEGs) between CMSs (|log₂FC|>1, P<0.001, Supplementary FIG. 13A, Supplementary Table 22), where 147 and 169 genes were upregulated and downregulated, respectively, in subtype 2 compared to subtype 1. Gene ontology (GO) functional enrichment analysis of DEGs demonstrated significant enrichment (FDR<0.05) for biological processes involving hemostasis, keratinization, intermediate filament organization, humoral immune response, regulation of ERK1 and ERK2 cascade, positive regulation of acute inflammatory response (Supplementary FIG. 13B, Supplementary Table 23); Cellular component GO terms significantly enriched (FDR<0.05) in the DEGs included intermediate filament, blood microparticle, cluster of actin-based cell projections, collagen-containing extracellular matrix, and trans-Golgi network transport vesicle (Supplementary FIG. 13C, Supplementary Table 24), whereas molecular function GO terms (FDR<0.05) included structural constituent of cytoskeleton and cytokine activity (Supplementary FIG. 13D, Supplementary Table 25). KEGG analysis indicated that DEGs were significantly enriched (FDR<0.05) in neuroactive ligand-receptor interaction, cytokine-cytokine receptor interaction, IL-17 signaling pathway, complement and coagulation cascades, and Staphylococcus aureus infection (Supplementary FIG. 13E, Supplementary Table 26). Moreover, protein-protein interaction (PPI) network analysis suggested that 72 genes with a degree no less than 5 were at the hub of the network (Supplementary Table 27, Supplementary FIG. 14). Together these findings suggest possible differences in the molecular mechanisms of CMSs.

Association of CMSs with Tumor Immune Microenvironment

Based on the molecular annotation of DEGs between CMSs, we investigated their association with the immune microenvironments. Subtype 2 (FIG. 5, Panel A) showed significantly more infiltrating B cells (P=0.027), dendritic cells (P=0.024), eosinophils (P=0.033), macrophages (P=0.02), mast cells (P=0.0034), natural killer (NK) cells (P=0.01), neutrophils (P=0.025), gamma delta T cells (P=0.0097), T regulatory cells (P=0.0042), macrophages M1 (P=0.003), and monocytes (P=0.029) compared to subtype 1. There was a trend toward increased abundance of CD4⁺ T cells (P=0.065), CD8⁺ T cells (P=0.057), and plasma cells (P=0.072) in subtype 2. Moreover, the T-cell infiltration score (P=0.00097) and overall immune infiltration score (P=0.029) were significantly higher in subtype 2 (FIG. 5, Panel A). Importantly, we validated the immune infiltrations in the ZN-LGG cohort using IHC (FIG. 5, Panel B, Supplementary FIG. 15), and confirmed the significantly more infiltrating T cells (CD3+) (P=1.3E-6), B cells (CD20+) (P=0.00042), and macrophages M1 (CD80+) (P=0.037) in subtype 2 patients. In addition, no statistical difference of macrophages M2 (CD163+) (P=0.57) between CMSs was found.

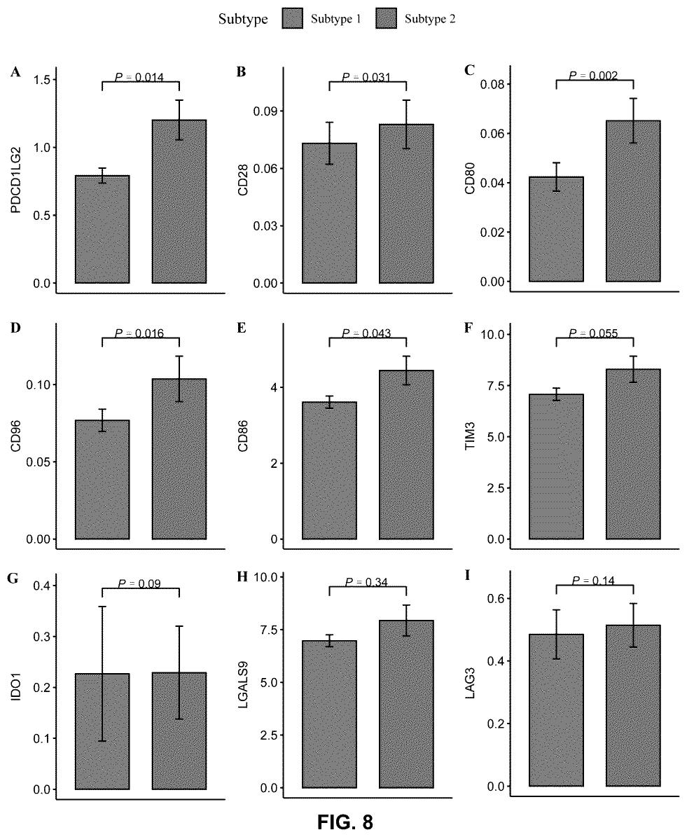

To explore the possibility of immune escape in subtype 2 LGG patients, we examined expression levels of immune suppression molecules CTLA-4, PD-1, the ligand of PD-1 (i.e., PD-L1), HAVCR2, LGALS9, CD86, LAG3, PDCD1LG2, CD28, CD96, CD80, and IDO1. In TCGA-LGG (FIG. 6, Panel A, FIG. 8), the expression of PD-1 (P=0.00044), PD-L1 (P=0.03), PDCDILG2 (P=0.014), CD96 (P=0.016), CD28 (P=0.031), CD80 (P=0.002), and CD86 (P=0.043) were significantly higher in subtype 2 patients, with a similar trend for CTLA-4 (P=0.17), TIM3 (P=0.055), LGALS9 (P=0.34), LAG3 (P=0.14), and IDO1 (P=0.09). Finally, we validated the expression levels of these immune inhibitory molecular markers in ZN-LGG using IHC and confirmed significant upregulation of PD-1 (P=8e-05), PD-L1 (P=0.018), and CTLA-4 (P=0.00089) in subtype 2 (FIG. 6, Panels B and C). Overall, these results indicated possible mechanisms for immune escape or immune tolerance in subtype 2 tumors, which could explain the poor prognosis of subtype 2 patients and laid the foundation of potential immunotherapy for LGG patients.

### Discussion

In this study, we extracted CMBs from WSIs of LGG patients through unsupervised learning strategy and subsequently defined two CMSs. Different from classical biomarkers, the CMBs act as imaging biomarkers capturing the heterogeneity in cellular properties and their microenvironments, which could be further explored as a future direction. The robustness of CMSs was demonstrated in two independent LGG cohorts. Interestingly, although a minority of GBM arises through the progression from LGG, the relevance of CMSs from LGG was shown to have prognostic value in GBM in two independent GBM cohorts, possibly related to common tumor microenvironments between LGG and GBM captured in CMSs. Although the HR of CMS was not as large as the HRs of well-known prognostic factors in gliomas (e.g., grade, IDH mutation status), the importance of CMSs lies in its independent prognostic significance after adjusting for other clinical and molecular factors; the relation to immunosuppressive tumor microenvironments; the association with treatment response; and the relation to underlying molecular and phenotypic alterations.

Different from many CNN-like systems, which mainly focus on end-to-end prediction of clinical/molecular endpoints, the emphasis of our study was on novel knowledge discovery with interpretability, robustness, and independent clinical value through multicentric validation. As a further justification, we evaluated a superior CNN-like system (i.e., SCNN [survival CNN]), specifically designed and optimized for the prediction of cancer outcomes in brain tumor.²² Interestingly, the SCNN risk score did not provide independent and significant prognostic value in both TCGA-LGG (P=0.182, Supplementary FIG. 17A) and TCGA-GBM (P=0.533, Supplementary FIG. 17B) cohorts, in the presence of CMS and other important clinical/molecular factors, suggesting that CMS out-performed the supervised CNN-like system (i.e., SCNN) for precision prognosis.

SCNA score, closely related to the occurrence and progression of many tumors (including glioma), is related to poor prognosis.²³ Meanwhile, TMB levels, closely related to degree of malignancy and poor prognosis of glioma, are often used as a biomarker for predicting the efficacy of anti-PD-1 therapy.^{24,25} Our study confirmed significantly higher focal SCNA scores and TMB levels in subtype 2 patients, which explains the poor prognosis and provides justification for anti-PD-1 immunotherapy for subtype 2 patients.

Our KEGG analysis suggested that DEGs were significantly enriched (FDR<0.05) in neuroactive ligand-receptor interaction, cytokine-cytokine receptor interaction, IL-17 signaling pathway, complement and coagulation cascades, and S. aureus infection, which were closely associated with the diagnosis and/or prognosis of glioma.²⁶⁻³⁰ Moreover, IL-6, at the hub of the PPI network (Supplementary FIG. 14), was recognized as an indicator for the oncogenesis, invasiveness, prognosis, and treatment of patient with glioma.³¹⁻³³ In addition, through oncoKB database, we found that MET (mesenchymal-epithelial transition, one of the hub DEGs), as a receptor tyrosine kinase, was selected as a target for various drugs in lung cancer, such as Capmatinib, Tepotinib, Capmatinib, and Tepotinib, etc. Together, these findings explained the prognostic role and treatment implications of CMS in glioma at the molecular level (detailed discussion refer to Supplementary Discussion 1).

The tumor immune microenvironment plays an important role in tumor progression. In glioma, NK cells, macrophages, neutrophils, CD4⁺ T cells, CD8⁺ T cells, regulatory T cells, etc. influence disease outcome.³⁴ Molinaro et al³⁵ evaluated immune cell fractions and epigenetic age in glioma patients and found that IDH/1p19q/TERT-WT patients had lower lymphocyte fractions (CD4⁺ T, CD8⁺ T, NK, and B cells) and higher neutrophil fractions than people without glioma, suggesting that common host immune factors among different glioma types may affect survival. Consist with previous studies, we showed that T cells (including CD4⁺ T cells, CD8⁺ T cells, gamma delta T cells, regulatory T cells), B cells, plasma cells, macrophages, NK cells, neutrophils, mast cells, etc. were higher in subtype type 2 patients, suggesting higher immune infiltration in tumors of subtype 2 patients. Moreover, we examined expression levels of immune inhibitory receptor CTLA-4 and PD-1 and the ligand of PD-1 (i.e., PDCD1L1), HAVCR2, LGALS9, CD86, LAG3, PDCD1LG2, CD28, CD96, CD80, and IDO1. The expression levels of these immune suppression molecules (FIG. 6, Panel A, FIG. 8) were significantly or tend to be significantly higher in the poor-prognosis subtype.

CTLA-4 inhibits T-cell activation by inducing antigen-presenting cells to express CD80 and CD86.6.³⁶ Regulatory T cells can inhibit T-cell function by secreting IL-10 and TGF-β.³⁷ Studies have reported that neutrophil infiltration in tumor tissues can promote tumor progression and metastasis, and in glioma, neutrophils can promote tumor proliferation by inducing angiogenesis.³⁸⁻⁴⁰ NK cells are an important component of the human immune system. However, Poli et al showed that NK cells are in a state of inactivation in glioma.⁴¹ These results indicated possible mechanisms for immune escape or immune tolerance due to the influence of immunosuppressive cell (e.g., regulatory T cells) infiltration, T-cell function inactivation, and other factors in the poor subtype tumors, which could explain the poor prognosis of subtype 2 patients in spite of more immune cells enriched in this subtype. Given the role of these immunosuppressive molecules in cancer immunotherapy, CMS also lays the foundation to select patients for the targeted immunotherapy.³⁴ Surprisingly, there was no significant association between PIK3CA/PIK3R1 mutation or CDKN2A/B copy number alternation and CMBs (Supplementary FIGS. 18 and 19); also, no significant association between homologous recombination deficiency and CMS was identified (Supplementary FIG. 20), despite their clinical value in gliomas.^{42,43}

This study has some shortcomings. First, relatively few LGG patients were included in the validation cohorts, so the conclusions of this study need further verified in large-scale studies. Second, the prevalence of subtype 2 was potentially due to the differences in patient population across hospitals. Nevertheless, our findings demonstrated the robustness and significant clinical value of CMS in all five cohorts. However, further large-scale studies are still needed to evaluate the impact of population difference on CMS before its utility in clinical practice. Third, our findings raise the possibility that subtype 2 LGG patients could benefit from anti-PD-1 immunotherapy; however, since LGG patients have not been recommended for anti-PD-1 immunotherapy based on existing clinical practice, we could not find any retrospective dataset to test this and will investigate it in our future prospective study.

In conclusion, we developed a pathology image-based LGG subtyping that seems to stratify LGG patients into two groups with different OS associated with treatment responses, copy number alterations, and TMB levels and immune tolerance. It provides a cost-effective solution with potential applicability worldwide in current clinical settings (Supplementary Table 28).

Supplementary Method 1. Cellular Morphometric Feature Estimation. The nuclear size was calculated based on segmented nuclear region; the Cellular Voronoi Size was calculated based on the voronoi region, which is the pixel set that is closest to a specific segmented nuclear region; the aspect ratio, major axis, minor axis and rotation were estimated based on the ellipse fitted from segmented nuclear contour; the curvature related features (e.g., bending energy, STD curvature, Abs max curvature) were estimated based on the curvature values along segmented nuclear contour¹; the intensity based features were estimated in gray scale in segmented nuclear region and its background (i.e., area that is outside nuclear region, and inside the corresponding voronoi region); and gradient related features were estimated using the first derivative of gaussian.

Supplementary Method 2. Clinical Evaluation and Validation of Patient Subtype. We evaluated and independently validated the clinical impact of pre-identified patient subtype from TCGA-LGG cohort, ZN-LGG cohort, SU-LGG cohort, TCGA-GBM cohort, and ZN-GBM cohort, respectively, where the latest clinical data of TCGA-LGG and TCGA-GBM cohorts was downloaded from Genomic Data Commons (GDC, https://portal.gdc.cancer.gov/), and the subtype assignment of each patient in independent validation cohorts (i.e., ZN-LGG, SU-LGG, TCGA-GBM, and ZN-GBM) was achieved through the application of pre-built TCGA-LGG patient subtype model as described previously. The evaluation and validation reside in three folds as follows, (1) Prognostic impact. The prognostic impact of patient subtype on OS was evaluated on TCGA-LGG, ZN-LGG, SU-LGG, TCGA-GBM, and ZN-GBM cohorts with univariate and stepwise multivariate Cox proportional hazards regression (CoxPH) models (survival package in R, Version 3.2-3), and the subtype-specific survival was visualized through Kaplan-Meier curve (survminer package in R, Version 0.4.8); (2) Predictive power of survival. A nomogram, based on multivariate CoxPH model, was developed to assist the prediction of 3-year and 5-year survival rate of LGG patents, where the multivariate CoxPH model was constructed with selected variables (i.e., clinical factors, molecular factors, and patient subtype) based on their significant and independent prognostic impact. Specifically, during nomogram construction and validation, the patients in TCGA-LGG cohort were randomly partitioned into training set (60% patients) and testing set (40% patients) through stratified sampling strategy. Then, a nomogram was constructed (rms package in R, Version 6.0-1) on the training set to predict the 3-year, and 5-year overall patient survival. The performance of nomogram was evaluated based on concordance-index (C-index) with 1000 bootstraps on TCGA-LGG training set and test set, followed by calibration analysis to calibrate the performance of the nomogram; and (3) Treatment response. The treatment response was categorized as: Response (including complete remission and partial remission); and Non-response (including progressive disease and stable disease). And the differences in treatment response were assessed with Chi-square test for both primary therapy and follow-up treatment.

Supplementary Method 3. Differences in Gene expression, Mutation load, and Immune microenvironment between Subtypes. Differentially expressed genes (DEGs) between patient subtypes were estimated (edgeR package in R, Version 3.30.3) based on the count data of TCGA-LGG cohort, where genes with |log₂FC|>1 (FC: fold change) and P<0.001 were selected and visualized via volcano plot (EnhancedVolcano package in R, Version 1.6.0). Gene ontology (GO) and Kyoto Encyclopedia of Genes and Genomes (KEGG) pathway enrichment analysis were performed² (clusterProfiler package in R, Version 3.16.1) to exam the biological functions of DEGs. Moreover, we performed protein-protein interaction (PPI) network analysis on the DEGs using the String database (https://string-db.org/), and visualized the PPI network using R package igraph³. The total mutation number and somatic copy number alteration (SCNA) of each TCGA-LGG sample were calculated (maftool package in R, Version 2.4.05)⁴ on the basis of MuSe⁵ preprocessed mutation data. The SCNA levels of each patient in the TCGA-LGG cohort were calculated according to previous work⁶. The infiltration scores of 18 immune cells and overall immune infiltration score were estimated via R package “ConsensusTME” (version: 0.0.1.9000) 7, and total T cell infiltration score was calculated according to the method introduced by Senbabaoglu et al.⁸.

Supplementary Method 4. Immunohistochemical (IHC) Staining. IHC staining was carried out on 4-μm sections of formalin-fixed and paraffin-embedded tissues according to the standard protocol on the entire ZN-LGG cohort (70 patients in total). Briefly, sections were dewaxed and rehydrated in serial alcohol washes, and then the endogenous peroxidase activities were blocked. After the nonspecific sites were saturated with 5% normal goat serum, the sections were incubated overnight at 4° C. with anti-CD3 (Ready-to-Use, mouse mAb, #F7.2.38, Leica), anti-CD20 (Ready-to-Use, mouse mAb, #L26, Leica), anti-CD80 (Ready-to-Use, mouse mAb, #MRQ-26, Leica), anti-CD163 (1:500, rabbit mAb, #EPR1157(2), abcam), anti-PD-1 Ab (1:50, mouse mAb, #UMAB199, ZSGB-Bio), anti-PD-L1 Ab (1:100, rabbit mAb, #13684, Cell signaling), or anti-CTLA4 Ab (1:50, mouse mAb, #UMAB249, ZSGB-Bio), and then incubated with anti-rabbit or anti-mouse Ig secondary Ab. The sections were visualized with the biotin-peroxidase complex and were counterstained with hematoxylin. For the assessment of CD3, CD20, CD80, CD163, PD-1 and CTLA4, the stained sections were screened at low-power field (×40), and 5 hot spots were selected. The number of positive cells in these areas were counted at HPF×400, 0.47 mm². The expression of PD-L1 was scored as a percentage of tumor cells expressing PD-L1 (3, ≥50%; 2, ≥5% and <50%; 1, ≥1% and <5%; and 0, <1%), where the staining in areas of necrosis was not quantified. The assessment was conducted by two experienced neuropathologists blinded to clinical information.

Supplementary Method 5. Statistical Analysis. Survival differences between subtypes or groups were examined using log-rank test. Differences in the treatment response of primary therapy and follow-up treatment between subtypes were examined using Chi-square test. Differences in respect of the expression of four negative immune regulators CTLA4, PD-1 and PD-L1, the immune cell infiltration, and genomic heterogeneity (tumor mutation burden, somatic copy number alteration) between subtypes were analyzed with Mann-Whitney non-parametric test. P value (FDR corrected if applicable) less than 0.05 was considered to be statistically significant. All analysis was performed with R (Version 4.0.2).

Supplementary Discussion 1. Extended discussion on gene function, pathway classifications and clinical relevance of DEGs. Our KEGG analysis suggested that DEGs were significantly enriched (FDR<0.05) in neuroactive ligand-receptor interaction, cytokine-cytokine receptor interaction, IL-17 signaling pathway, complement and coagulation cascades and Staphylococcus aureus infection. The pathway of neuroactive ligand-receptor interaction comprises of G-protein coupled receptors, ion channels and ligands which functions in modulation of neural plasticity, memory processes, behavior etc. Jagriti Pal et al. reported that defective neuroactive ligand receptor interaction pathway was a poor prognosticator in glioma patients⁹. Moreover, Xuemei Ji et al., by using eQTL analysis, suggested that the neuroactive ligand receptor interaction pathway was involved in lung cancer risk¹⁰. Cytokines are reported to be associated with host innate and adaptive inflammatory defenses, cell growth, differentiation, cell death, angiogenesis, and development and repair processes aimed at the restoration of homeostasis. Nijaguna et al. introduced an 18-cytokine signature that could be used for the diagnosis and prognosis for patients with glioma¹¹. The IL-17 signaling pathway mainly includes six members, IL-17A, IL-17B, IL-17C, IL-17D, IL-17E, and IL-17F, which are produced by multiple cell types and are involved in pro-inflammatory immune responses¹² and it was also reported to participated in the growth, progression and prognosis of glioma^{13,14}. Complement is an integral part of the immune system and mediates immune and inflammatory responses, classical pathway, lectin pathway and alternative pathway are reported to involved in glioma¹⁵. Moreover, the PPI network indicated that a total of 72 genes with a degree no less than 5 were at the hub of the network. As shown in Supplementary FIG. 14, IL-6, a soluble cytokine produced in response to inflammation, immune response, and hematopoiesis, is at the hub of the network, and it was recognized as an indicator for the oncogenesis, invasiveness, prognosis, and treatment of patient with glioma¹⁶⁻¹⁸. We further referenced the oncoKB database and found that IL6ST, an oncogene, acts as a signal transducer for IL-6 signaling, and the IL-6 cytokine binds to IL-6R, resulting in the homodimerization of IL6ST and the formation of IL-6/IL-6R Hexameric receptor complex 18. In addition, IL6ST is altered in inflammatory hepatocellular tumors primarily through in-frame deletions and missense mutations at the 1L-6/IL-6R binding site¹⁹. Moreover, we found that MET (mesenchymal epithelial transition), one of the hub DEGs, was recurrently altered by mutation, amplification and rarely altered by gene translocation in multiples cancers²⁰. As a receptor tyrosine kinase, MET was selected as a target for various drugs in lung cancer, such as Capmatinib, Tepotinib, Capmatinib and Tepotinib etc. In addition, other hub genes, i.e., CRP²¹, KNG1²², CCK^{23,24}, KRT family (KRT16, KRT14, KRT5, KRT15, KRT6B, KRT17, KRT84, KRT31, KRT1)²², CXCL1²⁵, PTGS2²⁶, HNF4A²⁷, LCN2²⁸, MET²⁹, SERPINA5³⁰, PAX2³¹, NTS³², and CHI3L1³³ were also reported to be involved in the growth and development of glioma. Thus, the above content explains the prognostic role and treatment implications of CMS in glioma at the molecular level.

TABLE 1 (Supplementary Table 6)
Description of cellular morphometric descriptors.

| Cellular Morphometric Descriptor | Description |
|---|---|
| Nuclear Size | Number of pixels of a segmented nuclear region |
| Cellular Voronoi Size | Number pixels of the voronoi region, where the segmented nucleus |
| | resides |
| Aspect Ratio | Aspect ratio of the segmented nucleus |
| Major Axis | Length of Major axis of the segmented nucleus |
| Minor Axis | Length of Minor axis of the segmented nucleus |
| Rotation | Angle between major axis and X axis of the segmented nucleus |
| Bending Energy | Mean squared curvature values along nuclear contour |
| STD Curvature | Standard deviation of absolute curvature values along nuclear contour |
| Abs Max Curvature | Maximum absolute curvature values along nuclear contour |
| Mean Nuclear Intensity | Mean intensity in nuclear region measured in gray scale |
| STD Nuclear Intensity | Standard deviation of intensity in nuclear region measured in gray scale |
| Mean Background Intensity | Mean intensity of nuclear background measured in gray scale |
| STD Background Intensity | Standard deviation of intensity of nuclear background measured in gray |
| | scale |
| Mean Nuclear Gradient | Mean gradient within nuclear region measured in gray scale |
| STD Nuclear Gradient | Standard deviation of gradient within nuclear region measured in gray |
| | scale |

TABLE 2
(Supplementary Table 22). Differentially expressed genes between Subtype 2
and Subtype 1 patients.

| ID | Gene | logFC | logCPM | LR | P Value | FDR |
|---|---|---|---|---|---|---|
| ENSG00000007908.14 | SELE | 1.658 | −0.292 | 34.136 | 5.14E−09 | 2.57E−06 |
| ENSG00000009709.10 | PAX7 | 1.806 | −0.460 | 51.995 | 5.56E−13 | 5.43E−10 |
| ENSG00000011083.7 | SLC6A7 | −1.128 | 2.341 | 14.930 | 1.12E−04 | 5.26E−03 |
| ENSG00000014257.14 | ACPP | 1.213 | −0.918 | 48.047 | 4.16E−12 | 3.76E−09 |
| ENSG00000016490.14 | CLCA1 | 1.062 | −4.881 | 15.397 | 8.71E−05 | 4.47E−03 |
| ENSG00000019169.10 | MARCO | −1.043 | 0.193 | 14.306 | 1.55E−04 | 6.56E−03 |
| ENSG00000034971.13 | MYOC | −2.200 | −2.062 | 33.570 | 6.87E−09 | 3.25E−06 |
| ENSG00000046604.11 | DSG2 | 1.438 | 0.236 | 26.597 | 2.51E−07 | 6.59E−05 |
| ENSG00000047936.9 | ROS1 | −1.314 | −2.021 | 16.833 | 4.08E−05 | 2.70E−03 |
| ENSG00000052850.5 | ALX4 | 1.748 | −0.016 | 94.026 | 3.11E−22 | 9.42E−19 |
| ENSG00000055732.11 | MCOLN3 | 1.326 | −1.937 | 43.504 | 4.23E−11 | 3.32E−08 |
| ENSG00000060566.12 | CREB3L3 | −1.021 | −2.872 | 14.007 | 1.82E−04 | 7.27E−03 |
| ENSG00000070748.16 | CHAT | 2.031 | −3.047 | 33.877 | 5.87E−09 | 2.82E−06 |
| ENSG00000073734.8 | ABCB11 | 1.165 | −3.580 | 21.219 | 4.10E−06 | 5.42E−04 |
| ENSG00000073756.10 | PTGS2 | 1.047 | 1.881 | 32.939 | 9.51E−09 | 4.32E−06 |
| ENSG00000075891.20 | PAX2 | 2.591 | −1.169 | 136.153 | 1.85E−31 | 1.12E−27 |
| ENSG00000077274.8 | CAPN6 | 1.533 | −3.300 | 40.046 | 2.48E−10 | 1.72E−07 |
| ENSG00000079689.12 | SCGN | −1.196 | −0.100 | 14.909 | 1.13E−04 | 5.30E−03 |
| ENSG00000088386.14 | SLC15A1 | 1.408 | −4.167 | 30.235 | 3.83E−08 | 1.47E−05 |
| ENSG00000091181.18 | IL5RA | 1.736 | −1.606 | 31.537 | 1.96E−08 | 8.28E−06 |
| ENSG00000091482.5 | SMPX | −1.168 | −2.656 | 17.335 | 3.13E−05 | 2.26E−03 |
| ENSG00000094796.4 | KRT31 | −1.827 | −2.466 | 37.120 | 1.11E−09 | 6.93E−07 |
| ENSG00000095596.10 | CYP26A1 | −1.035 | −1.519 | 34.785 | 3.68E−09 | 1.97E−06 |
| ENSG00000101076.15 | HNF4A | −1.056 | −3.093 | 13.507 | 2.38E−04 | 8.49E−03 |
| ENSG00000101292.7 | PROKR2 | −1.132 | −2.509 | 15.356 | 8.91E−05 | 4.53E−03 |
| ENSG00000101825.7 | MXRA5 | 1.018 | 2.805 | 32.303 | 1.32E−08 | 5.78E−06 |
| ENSG00000102195.8 | GPR50 | 2.409 | −2.777 | 81.031 | 2.22E−19 | 4.80E−16 |
| ENSG00000104321.9 | TRPA1 | 1.842 | −2.934 | 62.919 | 2.15E−15 | 3.10E−12 |
| ENSG00000104415.12 | WISP1 | 1.601 | 0.673 | 59.881 | 1.01E−14 | 1.30E−11 |
| ENSG00000104722.12 | NEFM | −1.160 | 5.330 | 17.464 | 2.93E−05 | 2.17E−03 |
| ENSG00000104938.15 | CLEC4M | −1.086 | −4.102 | 12.165 | 4.87E−04 | 1.35E−02 |
| ENSG00000105198.9 | LGALS13 | −3.239 | −4.475 | 19.225 | 1.16E−05 | 1.16E−03 |
| ENSG00000105398.3 | SULT2A1 | −1.215 | −4.868 | 11.996 | 5.33E−04 | 1.42E−02 |
| ENSG00000105825.10 | TFPI2 | 1.133 | −1.058 | 34.040 | 5.40E−09 | 2.66E−06 |
| ENSG00000105877.16 | DNAH11 | 1.038 | 0.161 | 24.314 | 8.19E−07 | 1.66E−04 |
| ENSG00000105976.13 | MET | −1.376 | 2.930 | 24.296 | 8.26E−07 | 1.67E−04 |
| ENSG00000106178.5 | CCL24 | 2.250 | −2.977 | 29.119 | 6.81E−08 | 2.41E−05 |
| ENSG00000106927.10 | AMBP | −1.417 | −3.418 | 26.363 | 2.83E−07 | 7.16E−05 |
| ENSG00000108342.11 | CSF3 | 2.238 | −1.727 | 71.842 | 2.33E−17 | 4.70E−14 |
| ENSG00000109132.6 | PHOX2B | 1.269 | −4.886 | 13.300 | 2.65E−04 | 9.15E−03 |
| ENSG00000109182.10 | CWH43 | 1.537 | −4.289 | 20.836 | 5.00E−06 | 6.30E−04 |
| ENSG00000109851.6 | DBX1 | 2.028 | −4.598 | 48.015 | 4.23E−12 | 3.76E−09 |
| ENSG00000110245.10 | APOC3 | −2.964 | −4.124 | 19.212 | 1.17E−05 | 1.16E−03 |
| ENSG00000111536.4 | IL26 | −1.390 | −4.724 | 16.036 | 6.22E−05 | 3.54E−03 |
| ENSG00000111863.11 | ADTRP | −1.114 | −1.372 | 27.051 | 1.98E−07 | 5.74E−05 |
| ENSG00000112238.11 | PRDM13 | 1.476 | −2.679 | 13.925 | 1.90E−04 | 7.45E−03 |
| ENSG00000112619.7 | PRPH2 | −1.072 | −0.015 | 26.603 | 2.50E−07 | 6.59E−05 |
| ENSG00000113430.8 | IRX4 | 1.218 | −3.888 | 22.825 | 1.77E−06 | 2.91E−04 |
| ENSG00000113889.10 | KNG1 | −1.257 | −3.436 | 15.095 | 1.02E−04 | 5.00E−03 |
| ENSG00000115705.19 | TPO | −1.241 | −3.580 | 27.741 | 1.39E−07 | 4.26E−05 |
| ENSG00000116690.10 | PRG4 | −1.503 | −0.672 | 42.951 | 5.61E−11 | 4.35E−08 |
| ENSG00000118194.17 | TNNT2 | −1.099 | 0.745 | 13.960 | 1.87E−04 | 7.35E−03 |
| ENSG00000120057.4 | SFRP5 | −1.095 | −0.298 | 17.884 | 2.35E−05 | 1.88E−03 |
| ENSG00000120093.10 | HOXB3 | 1.007 | 1.027 | 12.303 | 4.52E−04 | 1.28E−02 |
| ENSG00000120337.8 | TNFSF18 | 1.059 | 1.108 | 28.421 | 9.76E−08 | 3.28E−05 |
| ENSG00000121742.14 | GJB6 | −1.128 | 2.833 | 14.768 | 1.22E−04 | 5.56E−03 |
| ENSG00000122787.13 | AKR1D1 | 3.156 | −4.104 | 174.097 | 9.43E−40 | 1.14E−35 |
| ENSG00000122852.13 | SFTPA1 | 1.015 | −4.583 | 19.451 | 1.03E−05 | 1.07E−03 |
| ENSG00000123427.14 | METTL21B | 1.134 | 2.874 | 81.233 | 2.01E−19 | 4.49E−16 |
| ENSG00000124134.7 | KCNS1 | −1.049 | 2.533 | 12.142 | 4.93E−04 | 1.36E−02 |
| ENSG00000124157.6 | SEMG2 | −5.263 | −3.018 | 23.007 | 1.61E−06 | 2.73E−04 |
| ENSG00000124233.11 | SEMG1 | −5.405 | −2.371 | 21.410 | 3.71E−06 | 5.03E−04 |
| ENSG00000124490.12 | CRISP2 | 1.076 | −5.134 | 11.628 | 6.50E−04 | 1.62E−02 |
| ENSG00000124875.8 | CXCL6 | −2.092 | −1.210 | 33.399 | 7.51E−09 | 3.50E−06 |
| ENSG00000125522.3 | NPBWR2 | −1.440 | −3.769 | 13.165 | 2.85E−04 | 9.52E−03 |
| ENSG00000125726.9 | CD70 | 1.722 | −2.696 | 47.864 | 4.57E−12 | 4.00E−09 |
| ENSG00000125816.4 | NKX2−4 | −1.565 | −3.912 | 19.149 | 1.21E−05 | 1.19E−03 |
| ENSG00000125999.9 | BPIFB1 | −1.700 | −3.659 | 21.760 | 3.09E−06 | 4.51E−04 |
| ENSG00000126545.12 | CSN1S1 | 3.155 | −3.938 | 84.162 | 4.56E−20 | 1.10E−16 |
| ENSG00000127318.9 | IL22 | −1.813 | −5.100 | 11.526 | 6.86E−04 | 1.67E−02 |
| ENSG00000127329.13 | PTPRB | 1.753 | 5.171 | 209.968 | 1.40E−47 | 2.81E−43 |
| ENSG00000128422.14 | KRT17 | −1.972 | 0.673 | 51.086 | 8.84E−13 | 8.35E−10 |
| ENSG00000130182.6 | ZSCAN10 | −1.276 | −2.166 | 27.113 | 1.92E−07 | 5.58E−05 |
| ENSG00000130368.5 | MAS1 | −1.170 | 0.129 | 14.430 | 1.45E−04 | 6.27E−03 |
| ENSG00000130600.14 | H19 | 1.695 | 2.519 | 24.843 | 6.22E−07 | 1.33E−04 |
| ENSG00000131126.17 | TEX101 | 1.213 | −3.732 | 13.870 | 1.96E−04 | 7.58E−03 |
| ENSG00000131668.12 | BARX1 | 1.680 | −2.417 | 37.170 | 1.08E−09 | 6.82E−07 |
| ENSG00000131738.8 | KRT33B | −1.455 | −4.330 | 19.535 | 9.88E−06 | 1.03E−03 |
| ENSG00000131864.9 | USP29 | 1.711 | −4.462 | 31.486 | 2.01E−08 | 8.38E−06 |
| ENSG00000132693.11 | CRP | −2.671 | −4.507 | 13.864 | 1.97E−04 | 7.61E−03 |
| ENSG00000133048.11 | CHI3L1 | 1.102 | 8.042 | 13.365 | 2.56E−04 | 8.93E−03 |
| ENSG00000133110.13 | POSTN | 1.640 | 4.390 | 22.607 | 1.99E−06 | 3.19E−04 |
| ENSG00000133392.15 | MYH11 | 1.087 | 3.691 | 31.870 | 1.65E−08 | 7.12E−06 |
| ENSG00000133488.13 | SEC14L4 | −1.385 | −2.562 | 26.747 | 2.32E−07 | 6.23E−05 |
| ENSG00000133636.9 | NTS | 3.862 | 0.671 | 116.275 | 4.14E−27 | 1.67E−23 |
| ENSG00000133640.17 | LRRIQ1 | 1.225 | −0.378 | 24.324 | 8.14E−07 | 1.66E−04 |
| ENSG00000134389.9 | CFHR5 | −1.420 | −4.705 | 12.797 | 3.47E−04 | 1.08E−02 |
| ENSG00000134538.2 | SLCO1B1 | −2.365 | −2.977 | 22.250 | 2.39E−06 | 3.74E−04 |
| ENSG00000134757.4 | DSG3 | −1.188 | −3.357 | 10.909 | 9.57E−04 | 2.09E−02 |
| ENSG00000135426.13 | TESPA1 | 1.063 | 3.437 | 11.043 | 8.90E−04 | 1.99E−02 |
| ENSG00000136244.10 | IL6 | 1.282 | 0.190 | 29.580 | 5.37E−08 | 1.96E−05 |
| ENSG00000136535.13 | TBR1 | −1.038 | 2.871 | 17.473 | 2.91E−05 | 2.17E−03 |
| ENSG00000136542.7 | GALNT5 | 1.075 | −0.823 | 11.877 | 5.68E−04 | 1.48E−02 |
| ENSG00000137392.8 | CLPS | −1.073 | −4.550 | 12.053 | 5.17E−04 | 1.40E−02 |
| ENSG00000138083.4 | SIX3 | 1.401 | −1.803 | 32.718 | 1.07E−08 | 4.77E−06 |
| ENSG00000138472.9 | GUCA1C | −1.515 | −4.195 | 14.810 | 1.19E−04 | 5.50E−03 |
| ENSG00000139151.13 | PLCZ1 | 1.454 | −4.170 | 36.453 | 1.56E−09 | 9.27E−07 |
| ENSG00000139219.16 | COL2A1 | 1.725 | 0.858 | 52.225 | 4.95E−13 | 4.91E−10 |
| ENSG00000139304.11 | PTPRQ | 1.084 | −2.751 | 25.513 | 4.39E−07 | 1.03E−04 |
| ENSG00000139330.5 | KERA | 1.437 | −4.529 | 18.535 | 1.67E−05 | 1.47E−03 |
| ENSG00000140285.8 | FGF7 | −1.240 | −1.708 | 26.851 | 2.20E−07 | 6.07E−05 |
| ENSG00000140481.12 | CCDC33 | 1.328 | −1.192 | 21.412 | 3.70E−06 | 5.03E−04 |
| ENSG00000140798.14 | ABCC12 | −1.339 | −1.076 | 25.500 | 4.42E−07 | 1.03E−04 |
| ENSG00000142319.17 | SLC6A3 | 2.253 | −3.181 | 71.018 | 3.54E−17 | 6.69E−14 |
| ENSG00000142515.13 | KLK3 | 2.356 | −0.647 | 21.577 | 3.40E−06 | 4.84E−04 |
| ENSG00000142700.10 | DMRTA2 | 1.151 | 1.170 | 19.520 | 9.95E−06 | 1.04E−03 |
| ENSG00000143278.3 | F13B | 1.597 | −4.367 | 26.859 | 2.19E−07 | 6.07E−05 |
| ENSG00000143556.7 | S100A7 | −1.166 | −3.562 | 17.836 | 2.41E−05 | 1.91E−03 |
| ENSG00000145536.14 | ADAMTS16 | 1.220 | 0.299 | 60.117 | 8.94E−15 | 1.18E−11 |
| ENSG00000145863.9 | GABRA6 | −2.023 | −2.730 | 27.631 | 1.47E−07 | 4.49E−05 |
| ENSG00000146013.9 | GFRA3 | −1.320 | −2.097 | 22.077 | 2.62E−06 | 3.99E−04 |
| ENSG00000147571.4 | CRH | −1.009 | −0.633 | 15.576 | 7.93E−05 | 4.18E−03 |
| ENSG00000148346.10 | LCN2 | −1.611 | −2.487 | 18.427 | 1.77E−05 | 1.54E−03 |
| ENSG00000149305.5 | HTR3B | −1.098 | 1.588 | 11.033 | 8.95E−04 | 1.99E−02 |
| ENSG00000149742.8 | SLC22A9 | −1.185 | −3.429 | 16.807 | 4.14E−05 | 2.73E−03 |
| ENSG00000150175.13 | FRMPD2L2 | −1.071 | −1.254 | 18.907 | 1.37E−05 | 1.32E−03 |
| ENSG00000150244.11 | TRIM48 | −5.156 | −0.221 | 25.658 | 4.08E−07 | 9.72E−05 |
| ENSG00000151577.11 | DRD3 | 2.238 | −3.517 | 104.276 | 1.76E−24 | 6.26E−21 |
| ENSG00000153347.8 | FAM81B | 1.038 | 0.015 | 12.718 | 3.62E−04 | 1.11E−02 |
| ENSG00000153404.12 | PLEKHG4B | 1.288 | 0.254 | 41.223 | 1.36E−10 | 1.03E−07 |
| ENSG00000154146.11 | NRGN | −1.031 | 6.982 | 16.511 | 4.84E−05 | 3.00E−03 |
| ENSG00000154165.4 | GPR15 | 1.220 | −4.722 | 19.070 | 1.26E−05 | 1.24E−03 |
| ENSG00000154438.6 | ASZ1 | −2.591 | −4.675 | 12.262 | 4.62E−04 | 1.30E−02 |
| ENSG00000154760.12 | SLFN13 | 1.050 | 1.201 | 57.582 | 3.24E−14 | 3.85E−11 |
| ENSG00000154997.8 | 44088 | 8.464 | 1.445 | 635.159 | 3.77E−140 | 2.28E−135 |
| ENSG00000155495.8 | MAGEC1 | −2.647 | −4.034 | 17.976 | 2.24E−05 | 1.82E−03 |
| ENSG00000155761.12 | SPAG17 | 1.540 | −0.266 | 34.250 | 4.85E−09 | 2.46E−06 |
| ENSG00000156076.8 | WIF1 | −1.080 | 2.312 | 11.625 | 6.51E−04 | 1.62E−02 |
| ENSG00000157111.11 | TMEM171 | −1.329 | −1.417 | 28.021 | 1.20E−07 | 3.82E−05 |
| ENSG00000157765.10 | SLC34A2 | 1.096 | −0.161 | 20.377 | 6.36E−06 | 7.47E−04 |
| ENSG00000158816.14 | VWA5B1 | 1.168 | −1.588 | 34.375 | 4.54E−09 | 2.33E−06 |
| ENSG00000158874.10 | APOA2 | −3.836 | −2.174 | 48.301 | 3.66E−12 | 3.35E−09 |
| ENSG00000159251.6 | ACTC1 | 1.412 | 1.529 | 40.267 | 2.21E−10 | 1.58E−07 |
| ENSG00000159495.7 | TGM7 | −1.261 | −4.602 | 12.132 | 4.96E−04 | 1.36E−02 |
| ENSG00000160111.11 | CPAMD8 | −2.388 | 2.664 | 72.454 | 1.71E−17 | 3.57E−14 |
| ENSG00000160349.8 | LCN1 | −1.046 | −4.221 | 13.301 | 2.65E−04 | 9.15E−03 |
| ENSG00000160472.4 | TMEM190 | 1.661 | −3.581 | 39.526 | 3.24E−10 | 2.18E−07 |
| ENSG00000161849.3 | KRT84 | −2.123 | −4.158 | 23.807 | 1.07E−06 | 2.06E−04 |
| ENSG00000161905.11 | ALOX15 | 2.109 | −2.108 | 117.843 | 1.88E−27 | 8.11E−24 |
| ENSG00000162069.13 | CCDC64B | −1.033 | −2.854 | 23.551 | 1.22E−06 | 2.25E−04 |
| ENSG00000162598.12 | C1orf87 | 1.214 | −0.922 | 26.179 | 3.11E−07 | 7.72E−05 |
| ENSG00000163032.10 | VSNL1 | −1.011 | 6.334 | 14.169 | 1.67E−04 | 6.88E−03 |
| ENSG00000163263.6 | C1orf189 | 3.046 | −1.715 | 164.652 | 1.09E−37 | 1.10E−33 |
| ENSG00000163286.6 | ALPPL2 | 1.815 | −4.636 | 18.023 | 2.18E−05 | 1.80E−03 |
| ENSG00000163331.9 | DAPL1 | −1.092 | 1.717 | 18.595 | 1.62E−05 | 1.46E−03 |
| ENSG00000163646.9 | CLRN1 | 1.426 | −4.954 | 15.097 | 1.02E−04 | 5.00E−03 |
| ENSG00000163687.12 | DNASEIL3 | 1.072 | −1.117 | 25.834 | 3.72E−07 | 9.08E−05 |
| ENSG00000163739.4 | CXCL1 | 1.038 | 0.388 | 23.882 | 1.02E−06 | 1.99E−04 |
| ENSG00000163792.6 | TCF23 | 1.063 | −1.724 | 18.627 | 1.59E−05 | 1.45E−03 |
| ENSG00000163833.7 | FBXO40 | −1.196 | −1.484 | 23.927 | 1.00E−06 | 1.96E−04 |
| ENSG00000163914.4 | RHO | −1.111 | −2.858 | 24.273 | 8.36E−07 | 1.69E−04 |
| ENSG00000164093.14 | PITX2 | −2.643 | −1.162 | 28.611 | 8.85E−08 | 3.04E−05 |
| ENSG00000164363.9 | SLC6A18 | −1.695 | −3.569 | 16.698 | 4.38E−05 | 2.83E−03 |
| ENSG00000164509.12 | IL31RA | 1.048 | −3.547 | 13.964 | 1.86E−04 | 7.35E−03 |
| ENSG00000164600.5 | NEUROD6 | −1.327 | 1.349 | 17.089 | 3.57E−05 | 2.44E−03 |
| ENSG00000164879.6 | CA3 | 1.450 | 1.916 | 40.273 | 2.21E−10 | 1.58E−07 |
| ENSG00000165105.9 | RASEF | 1.430 | −0.092 | 53.561 | 2.51E−13 | 2.61E−10 |
| ENSG00000165553.4 | NGB | −1.191 | 1.149 | 17.141 | 3.47E−05 | 2.40E−03 |
| ENSG00000165643.9 | SOHLH1 | −1.199 | 0.697 | 21.281 | 3.97E−06 | 5.31E−04 |
| ENSG00000166961.13 | MS4A15 | −1.354 | −4.401 | 18.741 | 1.50E−05 | 1.39E−03 |
| ENSG00000167332.7 | OR51E2 | 1.033 | −3.357 | 17.468 | 2.92E−05 | 2.17E−03 |
| ENSG00000167434.8 | CA4 | 1.795 | 3.591 | 132.286 | 1.30E−30 | 7.12E−27 |
| ENSG00000167656.4 | LY6D | −2.252 | −2.989 | 26.948 | 2.09E−07 | 5.91E−05 |
| ENSG00000167749.10 | KLK4 | 1.145 | −3.757 | 21.880 | 2.90E−06 | 4.27E−04 |
| ENSG00000167751.11 | KLK2 | 2.047 | −2.143 | 25.003 | 5.72E−07 | 1.27E−04 |
| ENSG00000167768.4 | KRT1 | −1.041 | −3.434 | 14.676 | 1.28E−04 | 5.73E−03 |
| ENSG00000167916.4 | KRT24 | 1.991 | −4.946 | 30.996 | 2.59E−08 | 1.04E−05 |
| ENSG00000168334.8 | XIRP1 | 1.032 | −0.345 | 31.035 | 2.53E−08 | 1.03E−05 |
| ENSG00000168779.18 | SHOX2 | 1.210 | 1.116 | 14.130 | 1.71E−04 | 6.97E−03 |
| ENSG00000168878.15 | SFTPB | 1.742 | −3.351 | 91.950 | 8.89E−22 | 2.34E−18 |
| ENSG00000168907.12 | PLA2G4F | 2.170 | −4.531 | 70.070 | 5.72E−17 | 9.89E−14 |
| ENSG00000169344.14 | UMOD | 2.325 | −5.121 | 44.170 | 3.01E−11 | 2.46E−08 |
| ENSG00000169435.12 | RASSF6 | 3.397 | −3.210 | 147.495 | 6.12E−34 | 4.54E−30 |
| ENSG00000170439.6 | METTL7B | 1.122 | 4.033 | 27.894 | 1.28E−07 | 3.95E−05 |
| ENSG00000170454.5 | KRT75 | −2.524 | −2.298 | 28.054 | 1.18E−07 | 3.78E−05 |
| ENSG00000170788.12 | DYDC1 | 1.218 | −4.980 | 13.458 | 2.44E−04 | 8.66E−03 |
| ENSG00000171346.12 | KRT15 | −1.302 | −2.641 | 15.377 | 8.81E−05 | 4.50E−03 |
| ENSG00000171401.13 | KRT13 | −2.010 | −1.654 | 14.340 | 1.53E−04 | 6.48E−03 |
| ENSG00000171501.8 | OR1N2 | −2.695 | −4.288 | 12.429 | 4.23E−04 | 1.22E−02 |
| ENSG00000171509.14 | RXFP1 | −1.115 | 1.333 | 22.605 | 1.99E−06 | 3.19E−04 |
| ENSG00000171517.5 | LPAR3 | −1.171 | −2.213 | 13.230 | 2.75E−04 | 9.34E−03 |
| ENSG00000171532.4 | NEUROD2 | −1.015 | 2.724 | 18.189 | 2.00E−05 | 1.70E−03 |
| ENSG00000171551.10 | ECEL1 | 1.415 | 2.030 | 30.200 | 3.90E−08 | 1.49E−05 |
| ENSG00000171557.15 | FGG | −2.138 | −4.047 | 12.996 | 3.12E−04 | 1.01E−02 |
| ENSG00000171564.10 | FGB | −1.955 | −4.051 | 17.763 | 2.50E−05 | 1.97E−03 |
| ENSG00000172238.4 | ATOH1 | 1.892 | −4.265 | 70.741 | 4.07E−17 | 7.25E−14 |
| ENSG00000172482.4 | AGXT | 1.520 | −4.035 | 23.473 | 1.27E−06 | 2.29E−04 |
| ENSG00000172782.10 | FADS6 | −1.018 | −0.434 | 13.291 | 2.67E−04 | 9.19E−03 |
| ENSG00000173110.7 | HSPA6 | 1.314 | 2.503 | 36.283 | 1.71E−09 | 1.00E−06 |
| ENSG00000173213.8 | RP11-683L23.1 | 3.135 | −2.654 | 149.355 | 2.40E−34 | 2.07E−30 |
| ENSG00000173714.7 | WFIKKN2 | −1.967 | 0.661 | 39.667 | 3.01E−10 | 2.05E−07 |
| ENSG00000174576.7 | NPAS4 | 1.429 | 0.811 | 22.245 | 2.40E−06 | 3.74E−04 |
| ENSG00000175084.10 | DES | 1.220 | 1.222 | 20.888 | 4.87E−06 | 6.18E−04 |
| ENSG00000175707.8 | KDF1 | −1.004 | −3.762 | 14.850 | 1.16E−04 | 5.41E−03 |
| ENSG00000176040.12 | TMPRSS7 | 1.511 | −2.176 | 35.479 | 2.58E−09 | 1.44E−06 |
| ENSG00000176194.16 | CIDEA | −1.163 | −1.546 | 19.936 | 8.01E−06 | 8.90E−04 |
| ENSG00000176601.10 | MAP3K19 | 1.097 | 0.575 | 18.730 | 1.51E−05 | 1.40E−03 |
| ENSG00000178363.4 | CALML3 | −1.630 | −3.440 | 20.762 | 5.20E−06 | 6.46E−04 |
| ENSG00000178773.13 | CPNE7 | −1.045 | 1.518 | 25.181 | 5.22E−07 | 1.19E−04 |
| ENSG00000178934.4 | LGALS7B | −1.686 | −4.675 | 19.788 | 8.65E−06 | 9.43E−04 |
| ENSG00000179420.11 | OR6W1P | −2.327 | −4.644 | 11.411 | 7.30E−04 | 1.75E−02 |
| ENSG00000179914.4 | ITLN1 | −3.957 | −2.814 | 30.773 | 2.90E−08 | 1.14E−05 |
| ENSG00000180347.12 | CCDC129 | −2.027 | 0.455 | 21.459 | 3.61E−06 | 4.98E−04 |
| ENSG00000181499.2 | OR6T1 | 4.895 | −3.971 | 214.575 | 1.38E−48 | 4.17E−44 |
| ENSG00000181541.5 | MAB21L2 | 1.388 | −2.299 | 21.547 | 3.45E−06 | 4.87E−04 |
| ENSG00000182111.8 | ZNF716 | −2.926 | −4.074 | 22.447 | 2.16E−06 | 3.42E−04 |
| ENSG00000182333.13 | LIPF | −2.657 | −2.902 | 13.300 | 2.65E−04 | 9.15E−03 |
| ENSG00000182759.3 | MAFA | 2.162 | −2.451 | 96.307 | 9.84E−23 | 3.13E−19 |
| ENSG00000184058.11 | TBX1 | 1.305 | −1.068 | 67.757 | 1.85E−16 | 2.94E−13 |
| ENSG00000185479.5 | KRT6B | −2.332 | −2.647 | 15.840 | 6.89E−05 | 3.82E−03 |
| ENSG00000185640.5 | KRT79 | −1.819 | −4.383 | 13.145 | 2.88E−04 | 9.57E−03 |
| ENSG00000185652.10 | NTF3 | 1.469 | −3.433 | 47.556 | 5.35E−12 | 4.62E−09 |
| ENSG00000185933.6 | CALHM1 | −1.336 | −1.134 | 25.096 | 5.46E−07 | 1.23E−04 |
| ENSG00000186081.10 | KRT5 | −1.148 | 0.522 | 16.715 | 4.34E−05 | 2.82E−03 |
| ENSG00000186471.11 | AKAP14 | 1.153 | −2.089 | 30.335 | 3.63E−08 | 1.41E−05 |
| ENSG00000186732.12 | MPPED1 | −1.126 | 3.484 | 19.378 | 1.07E−05 | 1.09E−03 |
| ENSG00000186832.7 | KRT16 | −2.191 | −1.676 | 23.244 | 1.43E−06 | 2.47E−04 |
| ENSG00000186847.5 | KRT14 | −2.747 | −0.983 | 25.556 | 4.30E−07 | 1.02E−04 |
| ENSG00000186897.4 | C1QL4 | 1.042 | 2.659 | 25.444 | 4.55E−07 | 1.06E−04 |
| ENSG00000187017.13 | ESPN | 1.943 | −0.534 | 119.333 | 8.86E−28 | 4.12E−24 |
| ENSG00000187094.10 | CCK | −1.052 | 3.925 | 16.702 | 4.37E−05 | 2.83E−03 |
| ENSG00000187492.7 | CDHR4 | 1.288 | −2.760 | 24.175 | 8.80E−07 | 1.76E−04 |
| ENSG00000187714.6 | SLC18A3 | 1.379 | −1.412 | 17.980 | 2.23E−05 | 1.82E−03 |
| ENSG00000187848.11 | P2RX2 | 1.512 | −1.443 | 32.600 | 1.13E−08 | 5.00E−06 |
| ENSG00000187942.10 | LDLRAD2 | 1.066 | −1.687 | 36.784 | 1.32E−09 | 8.06E−07 |
| ENSG00000188488.12 | SERPINA5 | −1.337 | 1.706 | 15.867 | 6.79E−05 | 3.78E−03 |
| ENSG00000188869.11 | TMC3 | −1.243 | −0.686 | 14.711 | 1.25E−04 | 5.68E−03 |
| ENSG00000196415.8 | PRTN3 | −1.076 | −2.780 | 15.619 | 7.75E−05 | 4.12E−03 |
| ENSG00000196805.7 | SPRR2B | −1.547 | −5.237 | 11.215 | 8.11E−04 | 1.88E−02 |
| ENSG00000197085.10 | NPSR1-AS1 | −1.073 | −2.063 | 15.093 | 1.02E−04 | 5.00E−03 |
| ENSG00000197587.9 | DMBX1 | 2.252 | −3.529 | 63.371 | 1.71E−15 | 2.53E−12 |
| ENSG00000198535.5 | C2CD4A | 1.151 | −1.612 | 28.422 | 9.76E−08 | 3.28E−05 |
| ENSG00000198744.5 | RP5-857K21.11 | −1.165 | 4.274 | 13.188 | 2.82E−04 | 9.46E−03 |
| ENSG00000198774.4 | RASSF9 | 2.284 | 1.317 | 93.130 | 4.90E−22 | 1.35E−18 |
| ENSG00000198788.8 | MUC2 | −1.801 | −3.354 | 14.813 | 1.19E−04 | 5.50E−03 |
| ENSG00000199289.1 | RNU6-502P | 2.089 | −5.265 | 21.714 | 3.16E−06 | 4.58E−04 |
| ENSG00000200198.1 | RN7SKP211 | 1.944 | −5.289 | 15.260 | 9.37E−05 | 4.72E−03 |
| ENSG00000200795.1 | RNU4-1 | −1.180 | −2.752 | 14.103 | 1.73E−04 | 7.04E−03 |
| ENSG00000202538.1 | RNU4-2 | −1.130 | −1.078 | 18.535 | 1.67E−05 | 1.47E−03 |
| ENSG00000203811.1 | HIST2H3C | −1.464 | −4.313 | 17.277 | 3.23E−05 | 2.30E−03 |
| ENSG00000204140.9 | CLPSL1 | −1.920 | −4.117 | 31.415 | 2.08E−08 | 8.63E−06 |
| ENSG00000204538.3 | PSORS1C2 | 1.225 | −3.862 | 24.815 | 6.31E−07 | 1.33E−04 |
| ENSG00000204612.1 | FOXB2 | 1.194 | −4.744 | 18.562 | 1.64E−05 | 1.47E−03 |
| ENSG00000204711.7 | C9orf135 | 1.338 | −2.720 | 36.591 | 1.46E−09 | 8.73E−07 |
| ENSG00000205899.3 | BHLHA9 | −1.151 | −3.408 | 13.946 | 1.88E−04 | 7.37E−03 |
| ENSG00000205922.4 | ONECUT3 | 1.905 | −2.785 | 99.523 | 1.94E−23 | 6.52E−20 |
| ENSG00000206075.12 | SERPINB5 | −1.328 | −3.914 | 13.490 | 2.40E−04 | 8.54E−03 |
| ENSG00000206192.7 | ANKRD20A9P | −1.356 | −5.052 | 10.832 | 9.98E−04 | 2.15E−02 |
| ENSG00000206623.1 | RNU6-979P | −1.686 | −5.043 | 11.737 | 6.13E−04 | 1.56E−02 |
| ENSG00000207611.1 | MIR149 | −1.548 | −5.010 | 23.612 | 1.18E−06 | 2.19E−04 |
| ENSG00000211892.3 | IGHG4 | 2.763 | −0.348 | 123.480 | 1.09E−28 | 5.52E−25 |
| ENSG00000211899.6 | IGHM | −1.213 | 1.898 | 12.471 | 4.13E−04 | 1.20E−02 |
| ENSG00000212932.3 | RPL23AP4 | 1.315 | −5.100 | 24.178 | 8.79E−07 | 1.76E−04 |
| ENSG00000213452.4 | AKRIB1P2 | −1.204 | −4.998 | 13.961 | 1.87E−04 | 7.35E−03 |
| ENSG00000213645.2 | SLC25A1P3 | −2.597 | −4.600 | 17.334 | 3.14E−05 | 2.26E−03 |
| ENSG00000213892.9 | CEACAM16 | 5.005 | −2.948 | 147.297 | 6.76E−34 | 4.54E−30 |
| ENSG00000213921.6 | LEUTX | 3.489 | −5.116 | 34.983 | 3.33E−09 | 1.81E−06 |
| ENSG00000214285.2 | NPS | −2.803 | −3.615 | 18.809 | 1.44E−05 | 1.36E−03 |
| ENSG00000216588.7 | IGSF23 | −1.637 | −3.725 | 23.502 | 1.25E−06 | 2.27E−04 |
| ENSG00000218772.2 | FAM8A6P | −1.148 | −4.800 | 10.951 | 9.36E−04 | 2.06E−02 |
| ENSG00000220113.2 | MTCYBP4 | −1.390 | −4.915 | 11.535 | 6.83E−04 | 1.67E−02 |
| ENSG00000220575.6 | HTR5A-AS1 | −1.000 | 0.167 | 10.965 | 9.29E−04 | 2.05E−02 |
| ENSG00000223518.5 | CSNK1A1P1 | 1.263 | −3.703 | 58.828 | 1.72E−14 | 2.12E−11 |
| ENSG00000223553.4 | SMPD4P1 | −1.799 | −2.602 | 22.932 | 1.68E−06 | 2.79E−04 |
| ENSG00000224792.5 | IQCF4 | −1.562 | −4.557 | 17.515 | 2.85E−05 | 2.14E−03 |
| ENSG00000225110.1 | LL0XNC01-16G2.1 | −1.009 | 0.462 | 13.234 | 2.75E−04 | 9.34E−03 |
| ENSG00000226025.8 | LGALS17A | 1.279 | −2.372 | 17.173 | 3.41E−05 | 2.37E−03 |
| ENSG00000226148.1 | SLC25A39P1 | 1.182 | −4.811 | 28.341 | 1.02E−07 | 3.40E−05 |
| ENSG00000226943.3 | ALG1L5P | 1.112 | −4.880 | 23.306 | 1.38E−06 | 2.43E−04 |
| ENSG00000227059.5 | ANHX | −1.416 | −4.826 | 12.206 | 4.76E−04 | 1.33E−02 |
| ENSG00000227300.11 | KRT16P2 | 1.413 | −3.848 | 14.000 | 1.83E−04 | 7.28E−03 |
| ENSG00000229604.2 | MTATP8P2 | −1.034 | −0.581 | 17.385 | 3.05E−05 | 2.22E−03 |
| ENSG00000229972.6 | IQCF3 | −1.588 | −4.644 | 21.420 | 3.69E−06 | 5.03E−04 |
| ENSG00000230873.7 | STMND1 | 1.058 | −3.655 | 18.671 | 1.55E−05 | 1.42E−03 |
| ENSG00000231475.3 | IGHV4-31 | −3.678 | −1.266 | 18.195 | 1.99E−05 | 1.69E−03 |
| ENSG00000231755.1 | CHODL-AS1 | 3.380 | −4.588 | 54.364 | 1.67E−13 | 1.83E−10 |
| ENSG00000232843.2 | SNX18P2 | 1.072 | −5.223 | 12.536 | 3.99E−04 | 1.18E−02 |
| ENSG00000233213.1 | KCNJ6-AS1 | 1.216 | −4.663 | 16.948 | 3.84E−05 | 2.57E−03 |
| ENSG00000233951.3 | RCC2P3 | 1.423 | −5.221 | 18.691 | 1.54E−05 | 1.41E−03 |
| ENSG00000234354.3 | RPS26P47 | 1.533 | −3.381 | 54.037 | 1.97E−13 | 2.09E−10 |
| ENSG00000235254.3 | TMEM185AP1 | −1.279 | −4.691 | 11.135 | 8.47E−04 | 1.93E−02 |
| ENSG00000236502.1 | SIX3-AS1 | 1.236 | −3.196 | 19.978 | 7.83E−06 | 8.73E−04 |
| ENSG00000236824.1 | BCYRN1 | −1.070 | 4.533 | 26.960 | 2.08E−07 | 5.90E−05 |
| ENSG00000236946.2 | HNRNPA1P70 | −1.802 | −4.554 | 17.304 | 3.19E−05 | 2.28E−03 |
| ENSG00000237547.1 | IGHJ2P | −4.091 | −4.549 | 10.893 | 9.65E−04 | 2.10E−02 |
| ENSG00000237691.1 | IFNWP2 | −1.084 | −4.275 | 13.256 | 2.72E−04 | 9.29E−03 |
| ENSG00000240194.5 | CYMP | −1.241 | −4.799 | 18.287 | 1.90E−05 | 1.64E−03 |
| ENSG00000241794.1 | SPRR2A | −2.151 | −4.246 | 14.353 | 1.52E−04 | 6.45E−03 |
| ENSG00000242524.1 | OR2U2P | 1.148 | −5.310 | 11.876 | 5.69E−04 | 1.48E−02 |
| ENSG00000242908.5 | AADACL2-AS1 | 1.169 | −5.173 | 14.964 | 1.10E−04 | 5.18E−03 |
| ENSG00000242990.2 | RPL13AP23 | −1.347 | −3.662 | 24.508 | 7.40E−07 | 1.52E−04 |
| ENSG00000243955.4 | GSTA1 | −1.132 | 4.169 | 12.100 | 5.04E−04 | 1.38E−02 |
| ENSG00000248550.3 | OTX2−AS1 | −2.024 | −4.293 | 12.709 | 3.64E−04 | 1.11E−02 |
| ENSG00000253267.4 | DLGAP2−AS1 | 1.159 | −5.194 | 15.504 | 8.23E−05 | 4.30E−03 |
| ENSG00000253569.1 | VENTXP5 | −1.414 | −4.899 | 11.659 | 6.39E−04 | 1.60E−02 |
| ENSG00000253709.1 | IGHV1-14 | 1.363 | −5.321 | 13.438 | 2.47E−04 | 8.71E−03 |
| ENSG00000255737.2 | AGAP2-AS1 | 1.283 | 1.408 | 46.158 | 1.09E−11 | 9.17E−09 |
| ENSG00000259234.4 | ANKRD34C-AS1 | −1.054 | −2.457 | 11.518 | 6.89E−04 | 1.68E−02 |
| ENSG00000259905.4 | PWRN1 | −1.312 | −2.346 | 20.825 | 5.03E−06 | 6.33E−04 |
| ENSG00000263639.4 | MSMB | 2.155 | −3.965 | 15.413 | 8.64E−05 | 4.45E−03 |
| ENSG00000265190.5 | ANXA8 | −1.017 | −4.178 | 17.300 | 3.19E−05 | 2.28E−03 |
| ENSG00000267313.5 | KC6 | −1.579 | 4.314 | 13.716 | 2.13E−04 | 7.92E−03 |
| ENSG00000269332.4 | GOLGA2P9 | −1.118 | −4.720 | 13.060 | 3.02E−04 | 9.87E−03 |
| ENSG00000273693.1 | C2orf27AP1 | −1.542 | −4.736 | 22.146 | 2.53E−06 | 3.88E−04 |
| ENSG00000273963.1 | ENPP7P14 | 3.521 | −4.009 | 198.506 | 4.42E−45 | 6.69E−41 |
| ENSG00000275385.1 | CCL18 | −1.844 | −0.643 | 19.021 | 1.29E−05 | 1.26E−03 |
| ENSG00000275722.3 | LYZL6 | 4.837 | −4.811 | 54.743 | 1.37E−13 | 1.54E−10 |
| ENSG00000275811.1 | HTR1DP1 | −1.305 | −4.686 | 12.297 | 4.54E−04 | 1.28E−02 |
| ENSG00000276399.1 | FLJ36000 | −2.416 | 4.764 | 15.363 | 8.87E−05 | 4.52E−03 |
| ENSG00000276715.3 | YWHAEP7 | 1.073 | −4.561 | 11.287 | 7.80E−04 | 1.83E−02 |
| ENSG00000277586.1 | NEFL | −1.096 | 6.308 | 15.353 | 8.92E−05 | 4.54E−03 |
| ENSG00000278195.1 | SSTR3 | −1.025 | 0.719 | 15.647 | 7.63E−05 | 4.09E−03 |
| ENSG00000278530.3 | CHMP1B2P | −1.325 | −3.668 | 12.588 | 3.88E−04 | 1.16E−02 |
| ENSG00000278771.1 | Metazoa-SRP | −1.273 | −2.393 | 26.439 | 2.72E−07 | 6.94E−05 |
| ENSG00000279516.1 | FAM230C | 1.721 | −4.922 | 11.539 | 6.81E−04 | 1.67E−02 |
| ENSG00000281591.1 | DBET | −1.633 | −3.705 | 23.795 | 1.07E−06 | 2.06E−04 |

### REFERENCES CITED HEREIN

1. Sidaway P. Low-grade glioma subtypes revealed. Nat Rev Clin Oncol. 2020; 17:335.
2. Sturm D, Pfister S M, Jones D T W. Pediatric gliomas: current concepts on diagnosis, biology, and clinical management. J Clin Oncol. 2017; 35:2370-2377.
3. Louis D N, Perry A, Wesseling P, Brat D J, Cree I A, Figarella-Branger D, et al. The 2021 WHO Classification of Tumors of the Central Nervous System: a summary. Neuro Oncol. 2021; 23:12.31-1251.
4. Louis D N, Perry A, Reifenberger G, von Deimling A, Figarella-Branger D, Cavenee W K, et al. The 2016 World Health Organization Classification of Tumors of the Central Nervous System: a summary. Acta Neuropathol. 2016; 131:803-820.
5. Keshri V, Deshpande R P, Chandrasekhar Y, Panigrahi M, Rao I S, Babu P P. Risk stratification in low grade glioma: a single institutional experience. Neurol India. 2020; 68:803-812.
6. Viaccoz A, Lekoubou A, Ducray F. Chemotherapy in low-grade glioras. Curr Opin Oncol. 2012; 24:694-701.
7. Sharma A, Graber Ji Overview of prognostic factors in adult gliomas, Ann Palliat Med 2021; 10:863-874.
8. Liang J, Lv X, Lu C, Ye X, Chen X, Fu J, et al. Prognostic factors of patients with gliomas—an analysis on 335 patients with glioblastora and other forms of gliomas. BMC Cancer 2020; 20:35.
9. Ceccarelli M, Barthel F P, Malta T M, Sabedot T S, Salama S R, Murray B A, et al. Molecular profiling reveals biologically discrete subsets and pathways of progression in diffuse glioma. Cell. 2016; 164:550-563.
10. Louis D N, Perry A, Reifenberger G, von Deimling A, Figarella-Branger D, Cavenee W K, et al. The 2016 World Health Organization Classification of Tumors of the Central Nervous System: a summary. Acta Neuropathol. 2016; 131:803-820.
11. Özcan H, Emiroğlu B G, Sabuncuoğlu H, Özdonan S, Soyer A, Saygi T. A comparative study for gliorna classification using deep convolutional neural networks. Math Biosci Eng. 2021; 18:1550-1572.
12. Ning Z, Luo J, Xiao Q, Cai L, Chen Y, Yu X, et al. Multi-modal magnetic resonance imaging-based grading analysis for gliomas by integrating radiomics and deep features. Ann Transl Med. 2021; 9:298.
13. Fukuma R, Yanagisawa T, Kinoshita M, Shinozaki T, Arita H, Kawaguchi A, et al. Prediction of IDH and TERT promoter mutations in low-grade glioma from magnetic resonance images using a convolutional neural network. Sci Rep. 2019; 9:20311,
14. Chang H, Han J., A D B, Loss L, Gray J, Spellman P, et al. Invariant delineation of nuclear architecture in glioblastoma multiforme for clinical and molecular association. IEEE Trans Med Imaging. 2013; 32:670-682.
15. Chang H, Borowsky A, Spellman P, Parvin B. Classification of tumor histology via morphometric context. Paper presented at: 2013 IEEE Conference on Computer Vision and Pattern Recognition. Portland, OR, USA, Jun. 23-28, 2013.
16. Chang H, Fontenay G V, Han J, Cong G, Baehner F L, Gray J W, et al. Morphometic analysis of TCGA glioblastoma multiforme. BMC Bioinforaitics. 2011; 12:484.
17. Chang H, Zhou Y, Borowsky A, Barner K, Spellman P, Parvin B. Stacked predictive sparse decomposition for classification of histology sections. Int J Comput Vis. 2015; 113:3-18.
18. Wolchok J D, Hoos A, O'Day S, Weber J S, Hamid O, Lebbé C, et al. Guidelines for the evaluation of immune therapy activity in solid tumors: immune-related response criteria. Clin Cancer Res. 2009; 15:7412-7420.
19. McKenna S J, Ricketts I W, Cairns A Y, Hussein K A. A comparison of neural network architectures for cervical cell classification. Paper presented at: 1993 Third International Conference on Artificial Neural Networks, Brighton, UK, May 25-27, 1993; 105-109.
20. Charles N A, Holland E C, Gilbertson R, Glass R, Kettenmann H. The brain tumor microenvironment. Glia. 2011; 59:1169-1180.
21. Zhou M. Restricted mean survival time and confidence intervals by empirical likelihood ratio. J Biopharm Stat. 2021; 31:362-374.
22. Mobadersany P, Yousefi S, Amgad M, Gutman D A, Barnholtz-Sloan J S, Velázquez Vega J E, et al. Predicting cancer outcomes from histology and genomics using convolutional networks. Proc Natl Acad Sci USA 2018; 115:E2970.
23. Petralia F, Tignor N, Reva B, Koptyra M, Chowdhury S, Rykunov D, et al. Integrated proteogenornic characterization across major histological types of pediatric brain cancer. Cell. 2020; 183:1962-1985.e31.
24. Marabelle A, Fakih M, Lopez J, Shah M, Shapira-Frommer R, Nakagawa K, et al. Association of tumour mutational burden with outcomes in patients with advanced solid tumours treated with pembrolizumab: prospective biomarker analysis of the multicohort, open-label, phase 2 KENNOTE-158 study. Lancet Oncol. 2020; 21:1353-1365.
25. Wang L, Ge J, Lan Y, Shi Y, Luo Y, Tan Y, et al. Tumor mutational burden is associated with poor outcomes in diffuse glioma. BMC Cancer. 2020; 20:213.
26. Pal J, Patil V, Kurmar A, KaurK, Sarkar C, Somasundaram K. Genetic landscape of glioma reveals defective neuroactive ligand receptor interaction pathway as a poor prognosticator in glioblastoma patients [abstract]. Paper presented at: Proceedings of the American Association for Cancer Research Annual Meeting 2017; Washington, DC; April 1-5. Philadelphia (PA): AACR; 2017.
27. Nijaguna M B, Patil V, Hegde A S, Chandramouli B A, Arivazhagan A, Santosh V, et al. An eighteen serum cytokine signature for discriminating glioma from normal healthy individuals. PLoS One. 2015; 10:e0137524.
28. Wang B, Zhao C H, Sun G, Zhang Z W, Qian B M, Zhu Y F, et al. IL-17 induces the proliferation and migration of glioma cells through the activation of PI3K/Akt1/NF-kappaB-p65. Cancer Lett. 2019; 447.93-104.
29. Parajuli P, Mittal S. Role of IL-17 in glioma progression. J Spine Neurosurg. 2013; Suppl 1:pii:S1-004.
30. Yarmnoska S K, Alawieh A M, Tomlinson S, Hoang K B. Modulation of the complement system by neoplastic disease of the central nervous system. Front Immunol 2021; 12:689435.
31. Shan Y, He X, Song W, Han D, Niu J, Wang J. Role of IL-6 in the invasiveness and prognosis of glioma. Int J Clin Med. 2015; 8:9114-9120.
32. Yang F, He Z, Duan H, Zhang), Li J, Yang H, et al. Synergistic immunotherapy of glioblastoma by dual targeting of IL-6 and CD40. Nat Commun. 2021; 12:3424.
33. Hibi M, Murakami M, Saito M, Hirano T, Taga T, Kishimoto T. Molecular cloning and expression of an IL-6 signal transducer, gp130. Cell, 1990:63:1149-1157.
34. Qi Y, Liu B, Sun Q, Xiong X, Chen Q. Immune checkpoint targeted therapy in glioma: status and hopes. Front Immunol 2020; 11:578877.
35. Molinaro A M, Wiencke J K, Warrier G, Koestler D C, Chunduru P, Lee J Y, et al. Interactions of age and blood immune factors and non-invasive prediction of glioma survival. J Natl Cancer Inst. 2021; 114:446-457.
36. Fong B, Jin R, Wang X, Safaee M, Lisiero D N, Yang I, et al. Monitoring of regulatory T cell frequencies and expression of CTLA-4 on T cells, before and after DC vaccination, can predict survival in GBM patients. PLoS One. 2012; 7:e32614.
37. Bettelli E Carrier Y, Gao W, Korn T, Strom T B, Oukka M, et al. Reciprocal developmental pathways for the generation of pathogenic effector TH17 and regulatory T cells. Nature. 2006; 441:235-238.
38. Khan S, Mittal S, McGee K, Alfaro-Munoz K D, Majd N, Balasubramaniyan V, et al. Role of neutrophils and myeloid-derived suppressor cells in glioma progression and treatment resistance. Int J Mol Sci. 2020; 21:1954,
39. Garrido-Navas C, de Miguel-Perez D, Exposito-Hernandez J, Bayarri C, Amezcua V, Ortigosa A, et al. Cooperative and escaping mechanisms between circulating tumor cells and blood constituents. Cells. 2019; 8:1382.
40. Fujita M, Scheurer M E, Decker S A, McDonald H A, Kobanbash G, Kastenbuber E R, et al. Role of type 1 IFNs in antiglioma immunosurveillance—using mouse studies to guide examination of novel prognostic markers in humans. Clin Cancer Res. 2010; 16:3409-3419.
41. Poli A, Wang J, Domingues O, Planaguma J, Yan T, Rygh C B, et al. Targeting glioblastoma with NK cells and mAb against NG2/CSPG4 prolongs animal survival. Oncotarget. 2013; 4:1527-1546.
42. Draaisma K, Wijnenga M M J, Weenink B. Gao Y, Smid M. Robe P, et al. PI3 kinase mutations and mutational load as poor prognostic markers in diffuse glioma patients. Acta Neuropathol Commun. 2015; 3:88.
43. Lu V M, O'Connor K P, Shah A H, Eichberg D G, Luther E M, Komotar R J, et al. The prognostic significance of CDKN2A homozygous deletion in IDH-mutant lower-grade glioma and glioblastoma: a systematic review of the contemporary literature. J Neurooncol, 2020:148:221-229.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for determining and treating a Lower Grade Glioma (LGG) subtype for a subject, the method comprising: (a) obtaining a tissue sample from a subject suffering from LGG, (b) determining a cellular morphometric subtype (CMS) and/or cellular morphometric biomarkers (CMBs) of the tissue sample, (c) identifying a LGG subtype of the subject as LGG subtype 1 or LGG subtype 2, and (d) treating the subject wherein (i) when the subject is LGG subtype 1, the subject is not treated with immunotherapy, and (ii) when the subject is LGG subtype 2, the subject is treated with immunotherapy.

2. The method of claim 1, wherein the immunotherapy is anti-PD-1, anti-PD-L1, and/or anti-CTLA-4 immunotherapy.
